SEQUENCE LISTING (12 sequences)

SEQ ID NO: 1 - DNA, 16 bp, Artificial Sequence
  Synthetic polynucleotide
  misc_feature (1)..(1): modified by thiol-6Sp
ctccctaataacaatt

SEQ ID NO: 2 - DNA, 15 bp, Artificial Sequence
  Synthetic polynucleotide
  misc_feature (15)..(15): modified by 6Sp-thiol
ttataactattccta

SEQ ID NO: 3 - DNA, 30 bp, Artificial Sequence
  Synthetic polynucleotide
taggaatagttataaattgttattagggag

SEQ ID NO: 4 - DNA, 15 bp, Artificial Sequence
  Synthetic polynucleotide
  misc_feature (1)..(1): modified by thiol-6Sp
gtgagtattcgtatg

SEQ ID NO: 5 - DNA, 15 bp, Artificial Sequence
  Synthetic polynucleotide
  misc_feature (15)..(15): modified by 6Sp-thiol
gtatctgatgtgaca

SEQ ID NO: 6 - DNA, 30 bp, Artificial Sequence
  Synthetic polynucleotide
tgtcacatcagataccatacgattactcag

SEQ ID NO: 7 - DNA, 15 bp, Artificial Sequence
  Synthetic polynucleotide
  misc_feature (1)..(1): modified by thiol-Sp6
cgctgttctcatgga

SEQ ID NO: 8 - DNA, 15 bp, Artificial Sequence
  Synthetic polynucleotide
  misc_feature (15)..(15): modified by Sp6-thiol
tgggatgccaagaac

SEQ ID NO: 9 - DNA, 25 bp, Artificial Sequence
  Synthetic polynucleotide
cggagactgtccattgtcccaggct

SEQ ID NO: 10 - DNA, 25 bp, Artificial Sequence
  Synthetic polynucleotide
gctgcgtgagctcggggactgtgag

SEQ ID NO: 11 - DNA, 15 bp, Artificial Sequence
  Synthetic polynucleotide
  misc_feature (15)..(15): modified by an 18-atom hexa-ethyleneglycol space and single thiol attached to 3′ end of the oligonulceotide
tgggatgccaagaac

SEQ ID NO: 12 - DNA, 12 bp, Artificial Sequence
  Synthetic polynucleotide
  misc_feature (1)..(1): modified by a single thiol attached to 5′ end of the oligonucleotide and an 18-atom hexa-ethyleneglycol spacer
aggaaggaagct

(19) United States
(12) Patent Application Publication
(10) Pub. No.: US 2021/0181202 A1
(43) Pub. Date: Jun. 17, 2021

(54) MOLECULAR DETECTION VIA PROGRAMMABLE SELF-ASSEMBLY

(71) Applicant: NanoBio Designs, LLC, Indianola, IA (US)

(72) Inventors: Derek M. Lyons, Altoona, IA (US); Aaron T. Santos, Indianola, IA (US)

(73) Assignee: NanoBio Designs, LLC, Indianola, IA (US)

(21) Appl. No.: 16/762,252

(22) PCT Filed: Nov. 9, 2018
(86) PCT No.: PCT/US2018/059944

§ 371 Date: May 7, 2020

Related U.S. Application Data
(60) Provisional application No. 62/584,286, filed on Nov. 10, 2017.

(51) Int. Cl. G01N 33/58; B22F 1/00; G01N 21/47; G01N 21/77; G01N 21/49; H01J 37/26; G01Q 60/24; G01N 21/64

(52) U.S. Cl. CPC G01N 33/587; B22F 1/0018; B22F 1/0044; B22F 1/0062; G01N 21/4788; G01N 21/77; B22F 2304/056; H01J 37/26; G01Q 60/24; G01N 21/6458; B22F 2301/10; B22F 2301/255; G01N 21/49

(57) ABSTRACT

The present invention pertains generally to the detection of molecules. In some embodiments, it pertains to the determination of molecules, qualitatively and/or quantitatively, using the assembly of nanoparticles into superstructures, e.g., with a predefined shape. In some embodiments, a sample comprising a target molecule to be determined, such as DNA, is exposed to a first nanostructure and a second nanostructure, which may be formed from one or more nanoparticles. In the presence of the target molecule, the first nanostructure and the second nanostructure may assemble, e.g., spontaneously, to form a molecule superstructure. In some cases, the molecular superstructures can be identified by some combination of optical microscopy and automated image processing. In other cases, the molecular superstructure is able to scatter or diffract light, such as visible or ultraviolet light. For example, in the presence of a target molecule, the superstructure may comprise a plurality of nanostructures in regular dynamic spacing, which may scatter or diffract light. By determining such light, the target molecule within the sample may be determined. Other embodiments are generally directed to such molecular superstructures, techniques for making or using such molecular superstructures, devices incorporating such molecular superstructures, or the like.

### RELATED APPLICATIONS

This application claims the benefit of U.S. Provisional Patent Application Ser. No. 62/584,286, filed Nov. 10, 2017, entitled “Molecular Detection via Programmable Self-Assembly,” by Lyons and Santos, incorporated herein by reference in its entirety.

### FIELD

The present invention pertains generally to the detection of molecules. In some embodiments, it pertains to the determination of molecules, qualitatively and/or quantitatively, using the assembly of nanoparticles into superstructures, e.g., with a predefined shape.

### BACKGROUND

Molecular detection using self-assembling nanoparticles have been previously described. These typically involve the colorimetric detection of aggregates that form in the presence of molecules of interest. Other methods use dielectric, paramagnetic, phosphorescent, or other properties to detect molecules of interest. However, these methods require the uncontrollable aggregation of nanoparticles into amorphous or undefined structures, thus limiting their detectability and usefulness. Accordingly, improvements in molecular detection are needed.

### SUMMARY

The present invention pertains generally to the detection of molecules. In some embodiments, it pertains to the detection of molecules via the programmable self-assembly of nanoparticles into superstructures, e.g., with a predefined shape. The subject matter of the present invention involves, in some cases, interrelated products, alternative solutions to a particular problem, and/or a plurality of different uses of one or more systems and/or articles.

In one aspect, the present invention is generally directed to a composition. The composition, in one set of embodiments, comprises a plurality of substantially identical first nanostructures each comprising a first plurality of nanoparticles joined by nucleic acids, a plurality of substantially identical second nanostructures each comprising a second plurality of nanoparticles joined by nucleic acids, and a plurality of target nucleic acids, at least some of which are immobilized to at least some of the plurality of first nanostructures and at least some of the plurality of second nanostructures to form a plurality of discrete, substantially identical molecular superstructures. In some instances, each molecular superstructure of the plurality of discrete, substantially identical molecular superstructures comprises a target nucleic acid immobilized to both a first nanostructure and a second nanostructure.

The composition, in another set of embodiments, comprises a plurality of substantially identical first nanostructures each comprising a first plurality of self-assembled nanoparticles, a plurality of substantially identical second nanostructures each comprising a second plurality of self-assembled nanoparticles, and a plurality of target molecules, at least some of which are immobilized to at least some of the plurality of first nanostructures and at least some of the plurality of second nanostructures to form a plurality of discrete, substantially identical molecular superstructures. In certain cases, each molecular superstructure of the plurality of discrete, substantially identical molecular superstructures comprises a target molecule immobilized to both a first nanostructure and a second nanostructure.

According to yet another set of embodiments, the composition comprises a first nanostructure comprising a first plurality of nanoparticles joined by nucleic acids, a second nanostructure comprising a second plurality of self-assembled joined by nucleic acids, and a plurality of target nucleic acids each immobilized to both the first nanostructure and the second nanostructure to form a molecular superstructure. In some embodiments, the molecular superstructure has a dynamic spacing between the first and second nanostructures of at least 200 nm.

The composition, in still another set of embodiments, comprises a first nanostructure comprising a first plurality of nanoparticles joined by nucleic acids, a second nanostructure comprising a second plurality of self-assembled joined by nucleic acids, and a plurality of target nucleic acids each immobilized to both the first nanostructure and the second nanostructure to form a molecular superstructure. In yet another set of embodiments, the composition comprises a first nanostructure comprising a first plurality of self-assembled nanoparticles, a second nanostructure comprising a second plurality of self-assembled nanoparticles, and one or more target molecules immobilized to both the first nanostructure and the second nanostructure to form a molecular superstructure.

Another aspect of the present invention is directed to a device. The device, in accordance with one set of embodiments, includes a substrate comprising a plurality of chambers, at least some chambers comprising a first nanostructure comprising a first plurality of nanoparticles joined by nucleic acids, a second nanostructure comprising a second plurality of self-assembled joined by nucleic acids, and a source of coherent light positioned to direct coherent light at at least one chamber of the plurality of chambers.

In yet another set of embodiments, the device comprises a substrate comprising a plurality of chambers, at least some chambers comprising a first nanostructure comprising a first plurality of nanoparticles joined by nucleic acids, a second nanostructure comprising a second plurality of self-assembled joined by nucleic acids, a source of light positioned to direct light at at least one chamber of the plurality of chambers, and a detector positioned to detect scattered and/or diffracted light from the at least one chamber in which the light from the source of light is directed.

The device, in still another set of embodiments, comprises a chamber comprising a first nanostructure comprising a first plurality of nanoparticles joined by nucleic acids, a second nanostructure comprising a second plurality of self-assembled joined by nucleic acids, and a source of coherent light positioned to direct coherent light at the chamber.

In another set of embodiments, the device comprises a chamber comprising a first nanostructure comprising a first plurality of nanoparticles joined by nucleic acids, a second nanostructure comprising a second plurality of self-assembled joined by nucleic acids, a source of light positioned to direct light at the chamber, and a detector positioned to detect scattered and/or diffracted light from the chamber.

In yet another aspect, the present invention is a method of determining molecules. In one set of embodiments, the method includes mixing a sample suspected of containing target molecules of interest with nanoparticles, allowing the molecule of interest and nanoparticles to self-assemble into predesigned molecular superstructures, and determining the pre-programmed superstructures.

According to another set of embodiments, the method comprises exposing a sample suspected of comprising a target molecule to a suspension comprising a first nanostructure comprising a first plurality of nanoparticles joined by nucleic acids, and a second nanostructure comprising a second plurality of nanoparticles joined by nucleic acids, and determining binding of the target molecule to both the first nanostructure and the second nanostructure. In some embodiments, binding of the target molecule to both the first nanostructure and the second nanostructure forms a molecular superstructure comprising the first nanostructure, the second nanostructure, and the target molecule.

The method, in still another set of embodiments, comprises exposing a sample suspected of comprising a target molecule to a suspension comprising a first nanostructure comprising self-assembled nanoparticles, and a second nanostructure comprising self-assembled nanoparticles, and determining binding of the target molecule to both the first nanostructure and the second nanostructure.

In yet another set of embodiments, the method comprises exposing a sample suspected of containing a target molecule to two or more nanoparticles that self-assemble with the target molecule to form a molecular superstructure comprising the two or more nanoparticles and the target molecule, and determining the molecular superstructure within the sample.

In still another aspect, the method is a method of forming a molecular structure. In some embodiments, the method includes connecting a first nanostructure comprising a first plurality of nanoparticles joined by nucleic acids to a second nanostructure comprising a second plurality of nanoparticles joined by nucleic acids using a target molecule able to bind to both the first nanostructure and the second nanostructure.

In another aspect, the present invention encompasses methods of making one or more of the embodiments described herein, for example, a self-assembled molecular superstructure. In still another aspect, the present invention encompasses methods of using one or more of the embodiments described herein, for example, a self-assembled molecular superstructure.

Other advantages and novel features of the present invention will become apparent from the following detailed description of various non-limiting embodiments of the invention when considered in conjunction with the accompanying figures.

### BRIEF DESCRIPTION OF THE DRAWINGS

Non-limiting embodiments of the present invention will be described by way of example with reference to the accompanying figures, which are schematic and are not intended to be drawn to scale. In the figures, each identical or nearly identical component illustrated is typically represented by a single numeral. For purposes of clarity, not every component is labeled in every figure, nor is every component of each embodiment of the invention shown where illustration is not necessary to allow those of ordinary skill in the art to understand the invention. In the figures:

FIG. 1 is a schematic diagram illustrating a sample target molecule of interest, with two subsequences A and B, in one embodiment of the invention;

FIG. 2 is a schematic diagram showing nanorods self-assembling to form ordered arrays in the presence of the target molecule of interest (e.g., genomic DNA), in another embodiment of the invention;

FIG. 3 illustrates an example of one embodiment, showing a sample of interest combined with the nanorods in a sample holder, where light from a source passes through the sample and is diffracted;

FIGS. 4A-4B illustrates another embodiment, showing a sample of interest (e.g., genomic DNA) combined with nanorods to form a molecular superstructure;

FIGS. 5A-5B illustrates yet another embodiment, showing (FIG. 5A) a microscope image of superstructures (in this instance comprised of nanorods), and (B) a processed version of the same image, in which single nanoparticles and nanoparticle superstructures (white) have been automatically detected and classified by a computer;

FIGS. 6A-6D illustrate examples of various molecular superstructures, in accordance with certain embodiments of the invention;

FIGS. 7A-7D illustrate various nanochains or other nanostructures, in certain embodiments of the invention;

FIGS. 8A-8B illustrate various thiol terminated oligonucleotide polymers, constituted of phosphate linked moieties, in accordance with certain embodiments of the invention;

FIG. 9 illustrate various thiol terminated oligonucleotide polymers, constituted of peptide linked moieties, in accordance with certain embodiments of the invention; and

FIGS. 10A-B show bright-field microscopy images captured of nanocubes that demonstrate the superstructure formed from parallel chains of nanocubes, in some embodiments of the invention.

### DETAILED DESCRIPTION

The present invention pertains generally to the detection of molecules. In some embodiments, it pertains to the determination of molecules, qualitatively and/or quantitatively, using the assembly of nanoparticles into superstructures, e.g., with a predefined shape. In some embodiments, a sample comprising a target molecule to be determined, such as DNA, is exposed to a first nanostructure and a second nanostructure, which may be formed from one or more nanoparticles. In the presence of the target molecule, the first nanostructure and the second nanostructure may assemble, e.g., spontaneously, to form a molecule superstructure. In some cases, the molecular superstructure is able to scatter or diffract light, such as visible or ultraviolet light. For example, in the presence of a target molecule, the superstructure may comprise a plurality of nanostructures in regular dynamic spacing, which may scatter or diffract light. By determining such light, the target molecule within the sample may be determined. Other embodiments are generally directed to such molecular superstructures, techniques for making or using such molecular superstructures, devices incorporating such molecular superstructures, or the like.

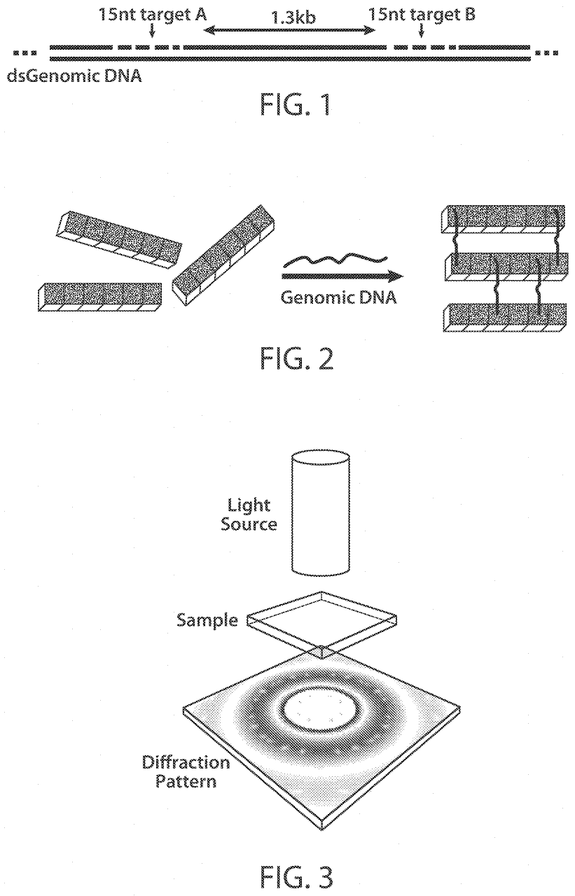

Certain aspects of the invention are generally directed to methods of detecting target molecules of interest via the self-assembly of nanoparticles into superstructures, and determining or identifying those superstructures. One non-limiting example is illustrated in FIG. 2. Consider a target molecule of interest to be a sequence of genomic DNA that one wishes to detect. Two subsequences of the genomic DNA are labeled A and B in FIG. 1. In this example, A and B are separated by 1.3 kb, and each of A and B is a sequence that is 15 nucleotides (nt) long (these numbers are arbitrarily chosen for illustrative purposes only). To detect this genomic DNA, one can utilize nanoparticles (in this example, nanocubes) to aid in the detection. In this example, one first assembles nanocubes into chains of nanocubes called nanochains. One or more portions of the nanochains may have “patches” or regions with a sequence of single-stranded DNA that is complimentary to a subsequence of the gene to be detected. (As discussed below, other methods of binding may be used in other embodiments.) Nanoparticles containing such patches may be prepared, for example, as discussed in Int. Pat. Apl. Pub. No. WO 2017/015444, incorporated herein by reference in its entirety.

In this example, at least two sets of nanochains are assembled. For illustration purposes, consider two nanochains whose sides are coated with oligonucleotides that contain respectively complementary sequences to either subsequence A or subsequence B from the genomic DNA. One may combine the nanochains with a sample matrix that may or may not contain the genomic sequence of interest. The sample may be purified to improve the performance, though this step is not necessary. When combined with a sample that contains the genomic sequence of interest, the subsequences A and B in the genomic DNA will hybridize to their respective compliments on the nanochains, forming a superstructure comprising arrays of nanochains with a predetermined spacing between chains (FIG. 2). For example, the spacing of the nanochains within the superstructure may be controlled based on the spacing between subsequences A and B. The superstructure will typically not stably form in the absence of the genomic sequence of interest.

It should be understood that the spacing between nanochains within the superstructure is not necessarily fixed, e.g., if the superstructure is “floating” in a suspension, but may vary somewhat in space or time as the components move about in suspension. However, there may be an average spacing (or “dynamic spacing”) between the nanochains, i.e., since the nanochains are “tied” together due to the genomic DNA, as is shown in FIG. 2. The average or dynamic spacing of nanochains within the nanostructure may be determinable, for example, based on light diffraction or scattering. Accordingly, this may be used to determine the presence or absence of the target molecules of interest within the sample, and/or the concentration of such target molecules.

In some instances, it may be beneficial to undergo a temperature cycle to aid the binding of the nanochains to the DNA, but this step is not necessary. In some instances, it is beneficial to dry the sample on a surface to enhance alignment of the arrays, but this step is also not necessary.

A variety of methods may be used to determine the molecular superstructure, e.g., qualitatively and/or quantitatively. The molecular superstructures may be contained within a sample, e.g., a solution or a suspension, a solid matrix, or the like. For example, in one embodiment, laser light may be directed towards the sample. If the superstructures are present, they may act as a diffraction grating (FIG. 3). Diffraction can then be detected, for example, by the presence of a diffraction pattern some distance from the incident beam. This may be measured by various light detectors such as a linear charge coupled device (CCD), which can identify the presence or absence of a diffraction pattern. As discussed below, however, other methods of determination may be used, including optical or microscopic techniques. In some cases, the unaided eye may be used; for example, samples containing such superstructures may appear to be “cloudy” if the superstructures are sufficiently large, while samples that lack such superstructures may not appear cloudy.

It should be understood, however, that the above example is by way of introduction only, and that in other embodiments, other types of nanoparticles, molecules of interest, superstructures, methods of determination, and the like may be used, as discussed in further detail herein. Briefly, non-limiting examples include the following.

A variety of target molecules may be determined in various embodiments, in addition to the example of genomic DNA described above. These include RNA, proteins, antibody-antigens, carbohydrates, polymers, and/or other biomolecules, etc.

In some embodiments, nanochains of nanoparticles may hydridize or otherwise bind to more than two molecule of interest. As a non-limiting example, the nanochains may specifically bind to more than two sub-sequences or portions of a polymeric molecule of interest, such as a nucleic acid or a polymer.

Nanoparticles as described herein are not limited to only nanocubes. Other shapes may be used in various embodiments including, for example, other faceted nanoparticles, nanorods, nanospheres, etc.

Nanochains or other nanostructures as described herein are typically assembled from nanoparticles. However, in some embodiments, the nanostructures are not assembled from nanoparticles, but may be formed as single entities. For example, in one set of embodiments, nanorods such as gold nanorods may be used as nanostructures.

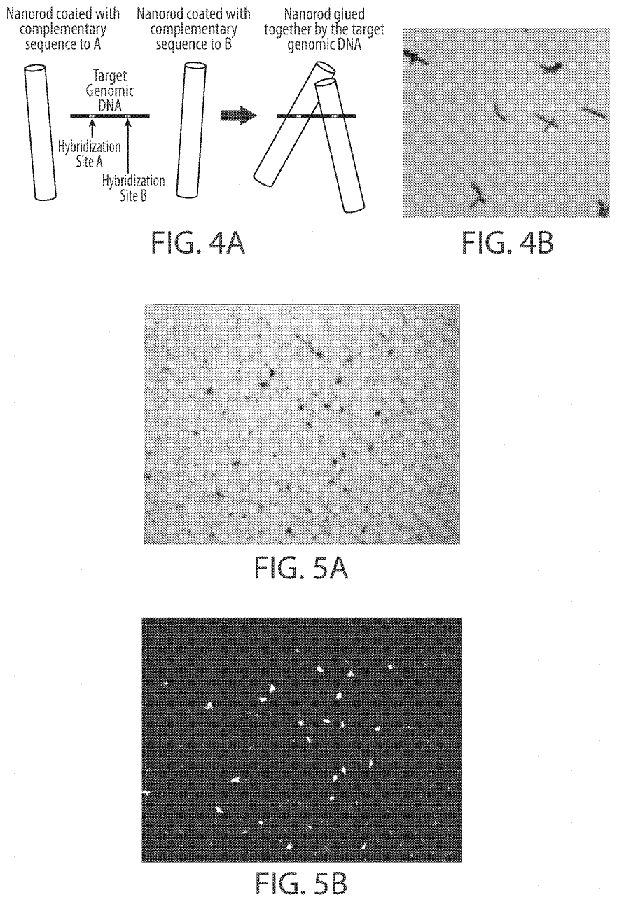

In another set of embodiments, the nanostructures such as nanochains may be selectively assembled together (or “glued”) in the presence of the a target molecule of interest, e.g., to form superstructures. As noted herein, the nanostructures may have a wide variety of shapes. An example of this is illustrated schematically in FIG. 4A. In this instance, the target molecule is genomic DNA with two target hybridization sites A and B. Two species of nanorods, one coated with single stranded DNA complementary to A and the other coated with single stranded DNA complementary to B, are combined in solution with a sample that may or may not contain the target genomic DNA sequence. If the target molecule is present in solution, the rods bind to form superstructures as illustrated schematically in FIG. 4A and as shown in the image in FIG. 4B. However, if the target molecule is not present, such superstructures are not able to form. Once formed, the presence and quantity of super structures can be determine in a variety of ways including, for example, optical microscopy or scattering, or other techniques such as those discussed herein.

Nanochains are typically formed from nanoparticles, which may linearly arranged to form the nanochains. However, it should be understood that linearly arranged nanoparticles are not required, and that it is possible, and in some cases may be advantageous, to bind the nanoparticles into shapes other than straight chains. Some non-limiting examples include topologically linear shapes (e.g., L-shapes, S-shapes, Z-shapes, zigzags, etc.), branching shapes, etc.

Bonding between the target molecules of interest and the nanochains was described above as hybridization between DNA molecules. However, other binding may occur in other embodiments, instead of (or in addition to) DNA hybridization. For example, bonding could occur via antibody-antigen or through selective covalent bonds, or using other mechanisms as discussed herein.

In some embodiments, the superstructures that are formed are substantially parallel arrays of nanochains. As noted above, the superstructures in some cases are not necessarily rigid, but may “float,” e.g., in suspension. However, in addition, other superstructure arrangements are also possible. For example, starting shapes of nanochains more complicated than nanorods may be used to produce more complicated final superstructures. This may, for example, produce more complicated superstructure arrangements, which may produce different diffraction and/or scattering patterns, which could be advantageous in certain instances.

The superstructure may have a variety of shapes, e.g., depending on the design of the nanoparticles such as nanocubes within the nanochains forming the superstructure. For example, in some cases, a target molecule may contains regions A′ and B′, which may bind respectively to faces (e.g., patches) A and B on different nanocubes (or other nanoparticles). By selecting the faces on which A and B appear, one can pre-program the shape of the final superstructure that may appear in the presence of the target molecules of interest. In some cases, for example, different target molecules may be used to produce superstructures with different shapes or dynamic spacings of nanochains, etc.

As noted, a variety of different methods of detecting light interactions with the sample may be used. For instance, the light source may include a visible light source, or other types of light sources such as ultraviolet light or infrared light sources may be used. The light source need not be a laser. For instance, it may be LED light, filtered sunlight, etc. In addition, other interrogation methods may be used instead of, or in addition to, light. Non-limiting examples include neutron or electron beams.

In addition, light (or other interrogation beams) need not pass through the sample. For instance, it may be advantageous in certain embodiments to detect light as it scatters off a surface (e.g., containing nanostructures or molecular superstructures), as in reflection.

A variety of methods may be used to determine light (or other interrogation methods) that interact with the sample. For example, diffraction may be determined in numerous ways including a CCD camera, a digital camera, an optical microscope, a fluorescent surface, one's own eye, etc. In addition, a variety of methods may be used for determination of target molecules, including detecting the scattering of electromagnetic waves (e.g. diffraction), optical detection (e.g. by eye or with an optical microscope), electron microscopy, atomic force microscopy, etc.

In some embodiments, the superstructures may be detected using microscopy, e.g., optical or electron microscopy. In certain cases, the presence or absence of a target molecule can be determined by the distribution of superstructures observed. For example, target molecules may selectively glue cause nanostructures to assemble together into form superstructures having various shapes (g., X-shapes, L-shapes, S-shapes, Z-shapes, zigzags, etc.). The distribution of superstructures may be used to determine whether a target gene molecule is present or absent, e.g. if the number of superstructures is greater than some threshold value, the target molecule is classified as present. In addition, in some cases, the concentration of the target molecules can be determined, e.g., by determining the aggregates. For example, the concentration of aggregates, the size of the aggregates, the speed at which aggregates form, etc., may be determined and used to determine the concentration of target molecules.

In some embodiments, image processing may be used to automate the identification of single nanoparticles and/or superstructures within a microscope image, e.g., as illustrated in FIG. 5A and FIG. 5B. For example, simple geometric features, e.g. length, width, etc., may be used to construct a rules-based engine to classify each object in the image as a single nanoparticle, a specific type of superstructure, or another type of structure. In some cases, one may, for example, techniques such as a neural networks or random forests may be used to classify the structure of each object in the image. Of course, in other embodiments, other techniques (including visual observation) may be used to determine superstructures.

In some embodiments, the number of target molecules present in the sample may be quantified, for example, by determining the number or concentration of single nanoparticles and/or the number or concentration of various types of superstructure that assemble in the presence or absence of target molecule. In some instances, image processing techniques may be used to automate the identification and counting of these the superstructures, e.g., if they have different structural morphologies. These techniques may, for example, determine not only that nanoparticles have bound to a target molecule, but also the number of bound particles and the distribution of superstructure shapes and sizes. In these cases, one may determine the amount and/or concentration of target molecules, e.g., by correlating amount to the distribution of self-assembled superstructures.

In some embodiments, it is possible to detect multiple molecules simultaneously, for instance, using multiple sets of nanostructures, e.g., which may be used to produce different diffraction patterns, or other effects.

As mentioned, some aspects of the present invention are generally directed to molecular superstructures that can be formed in the presence of a target molecule of interest. In some cases, the molecular superstructure may be formed from one or more nanoparticles assembled into nanochains or other nanostructures that are able to bind to one or more target molecules. In certain embodiments, there may be a generally repeating arrangement of nanochains within the molecular superstructure, which may interact with light, e.g., visible or infrared light, in some way, for example, causing diffraction, scattering, or other phenomena which may be determined to determine the target molecule.

In one aspect, the present invention is generally directed to molecular superstructures. Typically, molecular superstructures are formed from a plurality of molecules associated together into a coherent structure. For example, two or molecules forming the molecular superstructure may be associated together using hydrogen bonds, van der Waals forces, hydrophobic interactions, covalent coupling, physical entanglement, or the like. In some cases, the association may be via complementary nucleotide bonding (e.g., Watson-Crick pairing). In some cases, the molecular superstructure is “programmable” or predetermined, e.g., such that the structure of the molecular superstructure is determined based on an initial design of binding of the various molecules with each other (e.g., as opposed to random or spontaneous interactions). In some embodiments, a plurality of discrete, substantially identical molecular superstructures can be formed in a suspension, e.g., based on a predetermined design. It should be understood that such superstructures can be assembled as discrete entities, rather than being “unit cells” of an agglomerated “colloidal crystal” formed by the association of molecular superstructures to each other (i.e., the elements of the crystals are molecules, rather than atoms). Accordingly, a “discrete” entity is one that is not substantially bound to substantially identical copies of that entity. For instance, the discrete entities may be suspended within a liquid, where the discrete entities are not bound or agglomerated to each other, but instead are free to “float” in suspension, generally independently of the other discrete entities that are present. In contrast, entities that are not discrete may be associated or agglomerated with each other, e.g., as in a crystal or a colloidal crystal of molecules.

Thus, in certain embodiments, “building blocks” of molecules may associate together to build complex arbitrarily-shaped molecular superstructures via self-assembly or other techniques described herein. In some cases, the building blocks include nanostructures formed from nanorods, nanocubes, or other nanoparticles, which may be isolated or joined together to form nanochains or other suitable nanostructures, e.g., as discussed below. At least 2, at least 3, at least 4, at least 5, at least 7, at least 10, at least 15, or more nanostructures may be used to form the molecular superstructure in various embodiments. If more than one nanostructure is present, the nanostructures may be substantially the same, or different in some cases. For example, in some embodiments, the nanostructures have substantially the same size and/or shape. In certain cases, the nanostructures within a molecular superstructure are substantially identical except for having different binding regions or “patches,” e.g., as discussed below.

In addition, in certain cases, the nanostructures may be present within the molecular superstructure such that the nanostructures are substantially regularly spaced within the molecular superstructure. As previously mentioned, it should be understood that the spacing between nanochains within the molecular superstructure is not necessarily fixed, but may vary dynamically, e.g., in space and/or time. However, there may be an average or dynamic spacing between the nanostructures within the molecular superstructure, and this regular arrangement of nanostructures may be determinable, for example, using light diffraction or scattering, as discussed herein.

Thus, for example, the dynamic spacing between nanostructures such as nanochains within a molecular superstructure may be at least about 10 nm, at least about 20 nm, at least about 30 nm, at least about 50 nm, at least about 100 nm, at least about 200 nm, at least about 300 nm, at least about 400 nm, at least about 500 nm, at least about 600 nm, at least about 700 nm, at least about 800 nm, at least about 900 nm, at least about 1 micrometer, at least about 2 micrometers, at least about 3 micrometers, at least about 5 micrometers, at least about 10 micrometers, etc. In some cases, the dynamic spacing may be less than about 10 micrometers, less than about 5 micrometers, less than about 3 micrometers, less than about 2 micrometers, less than about 1 micrometers, less than about 900 nm, less than about 800 nm, less than about 700 nm, less than about 600 nm, less than about 500 nm, less than about 400 nm, less than about 300 nm, less than about 200 nm, less than about 100 nm, less than about 50 nm, less than about 30 nm, less than about 20 nm, less than about 10 nm, etc. In some cases, the dynamic spacing may be a combination of any of these, e.g., between about 300 nm and about 500 nm. In addition, in some cases, the dynamic spacing may be such that the molecular superstructure is able to diffract light, such as visible light (400 nm to 700 nm), infrared light (700 nm to 1 mm), and/or ultraviolet light (10 nm to 400 nm).

In addition, as discussed below, the nanostructures may include one or more binding sites or “patches” able to bind to one or more target molecules. In some cases, assembly of the nanostructures into molecular superstructure cannot occur without the target molecule of interest. For example, the target molecule may be a component of the molecular superstructure, or the target molecule may catalyze or otherwise facilitate placement of the nanostructures within the molecular superstructure. In some cases, more than one target molecule may be necessary to form a molecular superstructure. For example, the molecular superstructure may comprise at least 2, at least 3, at least 4, at least 5, at least 7, at least 10, at least 15, at least or 20 target molecules, e.g., each independently bound to one, two, three, or more of the nanostructures within the molecular superstructure. Not all target molecules need to bind to the same number of nanostructures within the molecular superstructure.

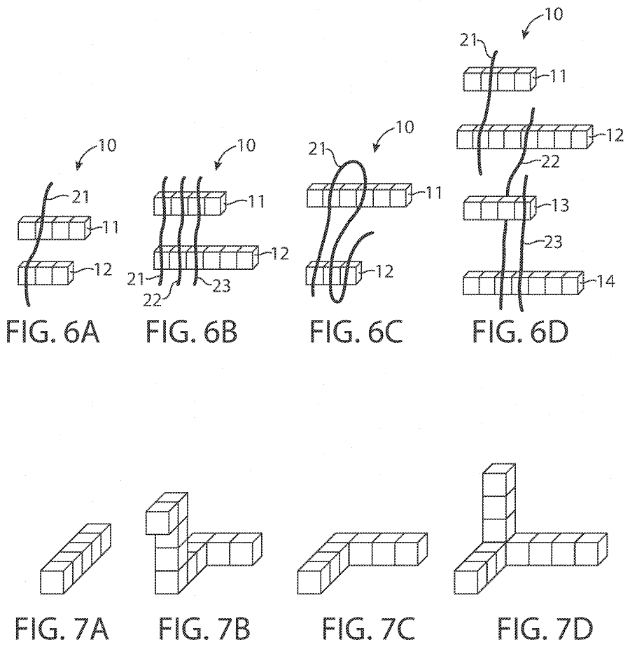

Non-limiting examples of various molecular superstructures can be seen in FIG. 6. In FIG. 6A, a molecular superstructure 10 is shown, comprising a nanostructures 11 and 12, and a target molecule 21 bound to both of the nanostructures. Nanostructures 11 and 12 may include one or more nanoparticles, such as nanocubes, or other nanoparticles such as those described herein. These may be formed into nanochains as shown in FIG. 6A, or other nanostructures. As shown in the example of FIG. 6A, a target molecule may in some embodiments specifically bind to a specific location of a nanostructure or portion thereof, such as a single face of a nanocube or other nanoparticle.

In some cases, more than one target molecule may be present within the molecular superstructure, e.g., as is shown in FIG. 6B with target molecules 21, 22, and 23 connecting nanostructures 11 and 12. Target molecules 21, 22, and 23 in this example may be the same, or different. In addition, in some cases, a target molecule may connect to a nanostructure at more than one location, e.g., as is shown in FIG. 6C.

Additionally, as another example, in some cases, a molecular superstructure may comprise 3, 4, 5, or more nanostructures. In some embodiments, the nanostructures may be substantially regularly arranged within the molecular superstructure, e.g., such that the molecular superstructure is able to scatter or diffract light. For instance, in FIG. 6D, nanostructures 11, 12, 13, and 14 are shown as being substantially regularly spaced within the molecular superstructure. However, it should be understood that in other embodiments, the nanostructures need not be substantially regularly arranged. In addition, it should be noted that in FIG. 6D, each of target molecules 21, 22, and 23 do not necessarily bind to each of nanostructures 11, 12, 13, and 14. Furthermore, it should be understood that the target molecules need not each bind to the same side of a nanostructure, e.g., as is shown with target molecules 22 and 23 in this figure.

The nanostructures within the molecular superstructure can take a wide variety of forms in different aspects of the invention. In one set of embodiments, the nanostructures may be formed of single entities, such as nanorods. For example, various nanorods and other nanostructures may be obtained commercially, for example, formed from metals such as gold, silver, copper, etc., carbon nanotubes, silicon or other semiconductor materials, or the like. Patches or other binding sites such as those discussed herein can be applied to various locations on such nanostructures.

However, in some embodiments, the nanostructures are formed from two or more nanoparticles, such as nanocubes, other faceted nanoparticles, nanorods, nanocylinders, nanospheres, etc. that are joined together. Non-limiting examples of such nanostructures may be seen in Int. Pat. Apl. Pub. No. WO 2017/015444, incorporated herein by reference in its entirety. In some cases, the nanoparticles are joined together to form the nanostructures using one or more binding regions or “patches” that can bind to another nanoparticle, in some cases specifically, using suitable binding partners such as DNA. For example, a face of a first nanocube (or other nanoparticle) may have a patch that is able to bind to a face of a second nanocube (or other nanoparticle), but is unable to bind to other faces of the second nanocube, or other nanocubes or nanoparticles. In this way, nanocubes and/or other nanoparticles may be assembled to form nanostructures in specific configurations, such as nanochains or other configurations described herein.

Many nanoparticle shapes can be used for the assembly of an ordered array of nanoparticles, e.g., in the presence of a target nucleotide sequence or other target molecule. Faceted nanoparticles, with more or less faces than nanocubes, can be used in addition to or instead of nanocubes in various embodiments. For example, oligonucleotides or other binding partners may be coated or otherwise present on the nanoparticle faces in a pattern that facilitates the assembly of nanoparticles into a nanochain, e.g., while exposing faces of the nanoparticle that are able to bind to a target molecule (for example, if a face was at least partially coated with oligonucleotides that are able to hybridize to nucleic acid target molecules). Spherical nanoparticles can be used in some embodiments, for example, if oligonucleotides or other binding partners are patterned on the nanoparticle surface in such a way as to provide assembly, e.g., while maintaining the ability to bind to the target molecule, such as a nucleotide sequence, using binding partners such as exposed, oligonucleotide-coated nanoparticle surfaces. As yet another example, nanorods can be assembled into ordered arrays in the presence of the target nucleotide sequences or other target molecules, for example, as polymeric chains or as monomers when the nanorod monomers are of sufficient length to produce ordered arrays in the presence of suitable target molecules. Examples of these are discussed in more detail below.

In one set of embodiments, the nanocubes (or other nanoparticles), may have one, two, three or more selectively binding chemical “patch” species that are on each face, and which may partially or completely cover a face. Typically, a “patch” will be present predominately on one face (or in some cases, more than one face), but will not be present in significant amounts on other faces. Some embodiments also may utilize such “patching” to assemble the nanostructures into molecular superstructures (for example, by binding to a target molecule), which can be used in a wide variety of applications, including those discussed herein.

The “building blocks” or nanoparticles that are assembled as discussed herein may have various advantages. For instance, some embodiments are directed to the self-assembly of arbitrarily-shaped molecular superstructures. These may be formed, in some cases, using the simple cubical shape of nanocubes and/or multiple selectively binding patches on various faces of the nanocubes or other nanoparticles, which may be, for example, face-centered, programmable, stackable, etc.

Incorporating cubical or other stackable geometry and a plurality of selectively binding patches may allow for the creation of nanochains or other nanostructures. For example, by incorporating more than two patches, programmability can be added, e.g., to allow the assembly of any arbitrary or designed nanostructure from a plurality of nanocubes or other nanoparticles. Patterned programmable selectively binding chemicals in patches on the nanoparticles may be achieved in some embodiments.

For instance, in some embodiments, programmability may allow one to pre-design the shape of the final target nanostructure or molecular superstructure. The geometry of the nanocubes or other nanoparticles may, in some cases, allow for face-to-face binding. The flat faces can be conjoined nearly parallel to each other, making designing target superstructures simple, because the nanoparticles can be bound flush against each other, and can be aligned on a straight-line rectangular grid, or in other predictable formats, depending on the nanoparticles. This geometry may permit the design and assembly of larger molecular superstructures, e.g., when considered in conjunction with suitable target molecules.

Thus, such programmability may allow a nanostructure to be defined in some cases on the basis of the ability of various nanoparticles to bind, e.g., in specific configurations or arrangements, thereby forming the nanostructure. Such design may occur in some cases even before the nanoparticles are synthesized. In some cases, such programmability may allow only one, or a relatively small number, of final nanostructures to be designed and assembled from the nanoparticles. For instance, after assembly, at least 50% or more of the nanostructures may share essentially identical configurations of nanoparticles that from the nanostructures.

Thus, the nanoparticles may include one or more “patches” on one or more faces in various embodiments, which can be used in the formation of nanostructures. For instance, a face of a nanoparticle may be modified with a chemical able to selectively bind other chemicals, e.g., attached to the faces of other nanoparticles. The face may thus be described as having a selectively binding chemical or a “patch.” The patches may then be used to assemble nanoparticles together into nanostructures.

Patches may be present on one or more faces of a nanoparticle, e.g., to 2, 3, 4, 5, 6, 7, 8, or more faces of a nanoparticle. The patches on each face of the nanoparticle may independently be the same or different. In addition, as discussed above, different nanoparticles may have different patches on them, e.g., to allow for the creation of more complex structures using nanoparticles. The patch may partially or completely cover the face of a nanoparticle.

At least some of the patches may be used to bind or attach the nanoparticles to other nanoparticles, e.g., to form a nanostructure of nanoparticles. The patches may be used to establish face-to-face binding or contact, e.g., between different nanoparticles, and the alignment of nanoparticles may be centered or off-centered in some cases. In some cases, the patches may be relatively unique, e.g., a patch may be able to specifically bind to only one (or a small number) of other patches within the nanostructure. Such specificity may allow only a small number of binding interactions between nanoparticles to occur, thereby allowing a specific nanostructure to form. For example, out of all of the binding interactions forming a nanostructure, each of the binding interactions may form no more than 50%, no more than 40%, no more than 30%, no more than 20%, no more than 10%, no more than 5%, or no more than 2% of all of the binding interactions that form the nanostructure. Different binding interactions may be non-interchangeable with each other, e.g., such that only certain combinations of binding partners (and thus, only certain nanoparticles are able to stably contact each other). In some cases, each binding interaction within a nanostructure of nanoparticles is unique.

As mentioned, a patch may independently cover all, or only a portion of, a face of a nanoparticle such as a nanocube. For instance, the patch may cover at least 10%, at least 20%, at least 30%, at least 40%, at least 50%, at least 60%, at least 70%, at least 80%, at least 90%, or substantially the entire face and/or no more than 90%, no more than 80%, no more than 70%, no more than 60%, no more than 50%, no more than 40%, no more than 30%, no more than 20%, or no more than 10% of the available surface area on the face of a nanoparticle such as a nanocube. Different faces of the nanocube may independently exhibit different amounts of coverage (or no coverage) by a patch, and different faces of a nanoparticle may exhibit the same or different patches, for instance, by being identical or different chemically, recognizing different binding partners, etc.

For brevity, some embodiments will be referred to herein as a “patching system,” though this is not meant to restrict the embodiments to one specific modality, as the associated devices and methods are also contemplated. Accordingly, in some embodiments of the invention, the disclosed patching methods segregate multiple selectively binding chemical patches on separate faces of nanocubes. “Patchy particles” (meaning particles on which at least one well-defined patch generates an anisotropic, directional interaction with other particles) can be used in certain embodiments.

In some cases, patches may be created by binding partners, which may be specific or non-specific. In some embodiments, a patch is able to only bind to one other specific patch within the nanostructure without being able to stably bind to other, incompatible patches within the nanostructure.

Because of its simple, sequence-dependent self-assembly characteristics, DNA is useful as a binding partner for a patch, e.g., as discussed herein. However, it should be understood that DNA is described here as one example, and other binding systems (or combinations of binding systems) may be used in other embodiments, such as discussed below. In some embodiments, for example, DNA can be segregated on the faces of a nanocube or other nanoparticle, which may simplify programmability or assembly, etc., as discussed herein.

The term “binding partner” or “binding chemical” generally refers to a molecule that can undergo binding with a particular partner, typically to a significantly higher degree than to other molecules, e.g., specific binding. For instance, the binding interaction between specific binding partners may be at least 10×, 100×, or 1000× greater than for any other binding partners that are present. In some cases, the binding between the binding partners may be essentially irreversible. Thus, for example, in the case of a receptor/ligand binding pair the ligand would specifically and/or preferentially select its receptor from a complex mixture of molecules, or vice versa. An enzyme would specifically bind to its substrate, a nucleic acid would specifically bind to its complement, an antibody would specifically bind to its antigen, etc. The binding interactions between binding partners may be, for example, hydrogen bonds, van der Waals forces, hydrophobic interactions, covalent coupling, or the like.

Thus, as other examples besides DNA hybridization (and/or hybridization of other nucleic acids), suitable patch systems include lock and key protein interactions such as avidin-biotin or enzyme-substrate interactions, antibody-antigen pairs, covalent coupling interactions, hydrophilic/hydrophobic/fluorinated interactions, and the like. Examples of some of these are discussed herein. As noted above, DNA may be particularly useful because of its simple programmable sequence-dependent binding rules, but the invention is not limited to only DNA patches. In addition, in some embodiments, more than one such system may be used, e.g., within the same patch, within different patches on the same nanoparticle, on different nanoparticles, or the like.

In one set of embodiments, different nucleic acid strands may be attached to various faces of a nanoparticle, which may be used to form unique patches on some or all of the faces of the nanoparticle. The nucleic acid strands may include, DNA, RNA, PNA, XNA, and/or any suitable combination of these and or other suitable polymers, and may comprise naturally-occurring bases and/or non-naturally-occurring bases. In some cases, due to the specificity of unique nucleic acid strands with each other, selective binding may be achieved between different patches on different nanoparticles. The nucleic acid strands may have any suitable number of nucleotides, and different patches may have nucleic acid strands with the same or different numbers of nucleotides. As non-limiting examples, the nucleic acid strands may include at least 6, at least 7, at least 10, at least 12, at least 15, at least 20, at least 25, at least 30, at least 35, at least 40, at least 45, at least 50, at least 55, at least 60, at least 70, at least 80, at least 90, or at least 100 nucleotides, which may be suitable produce a large number of relatively unique patches. As an illustrative example, using only the 4 naturally-occurring nucleotides, a DNA nucleic acid strand with 10 nucleotides would have 4¹⁰=1,048,576 combinations available (although not all of them need be used).

In some cases, conditions may be applied to facilitate binding or self-assembly, e.g., as discussed herein. For example, in one set of embodiments, heat may be applied to promote binding. A variety of techniques may be used to apply heat, including electrical resistance, Peltier elements, external heat sources, or the like. Thus, as a non-limiting example, a Peltier element may be used to heat a sample in the presence of the nanostructures to promote denaturation, e.g., of a DNA sequence. Once heated to a suitable temperature for a suitable length of time, the sampled may be cooled, for example, to promote hybridization of nanostructures to a target DNA sequence, which may cause assembly of the molecular superstructure.

In one set of embodiments, the miscibility of the patches within the nanoparticles may be different. Such miscibilities may be controlled, for example, by using moieties having different patterns of hydrophilicities/hydrophobicities. For instance, unique patches may be created on the faces of a nanoparticle using unique miscibilities on each face having a patch. Based on such miscibilities, binding partners having compatible miscibilities would be able to bind to the face while binding partners having incompatible miscibilities would be unable to bind to the face. In this way, unique patches may be created on some or all of the faces of the nanoparticle.

In some cases, miscibilities for the faces of a nanoparticle may be created using polymers having a variety of hydrophilic and/or hydrophobic groups, e.g., in a defined sequence. It should be understood that “hydrophilic” and “hydrophobic” groups are generally used in a relative sense with respect to miscibilities, i.e., hydrophilic groups generally prefer to associate with other hydrophilic groups rather than hydrophobic groups and vice versa, in such manner, a series of different hydrophilic groups and hydrophobic groups positioned within a polymer may define a miscibility for a polymer. It should also be understood that other interactions between hydrophilic/hydrophobic interactions may be used in other embodiments to define various miscibilities of a polymer; for example, such miscibilities may be defined by charged moieties within the polymer.

FIGS. 6-7 depict examples of embodiments of chemical structures of polymers comprising chemical moieties, for example, to control miscibilities. The polymers may be synthesized, for example, by chemically coupling monomers together to create patterns of chemical functionalities. The polymers in these examples may include a moiety (e.g. a thiol group) that bonds to the nanoparticle surface on one terminal end and a linker on the other end that displays chemically selective patch. For the sake of example, “B” in these figures may represent any of the five canonical nitrogenous bases found in nucleic acid polymers (i.e., adenine, thymine, cytosine, uracil, or guanine). “n” denotes the number of single monomer units that are repeated to build a polymer. “R” represents any type of chemical functionality used to provide chemical interactions between polymers. These examples represent the types of chemical functionalities useful for chemical interactions between polymers, but are not an inclusive list.

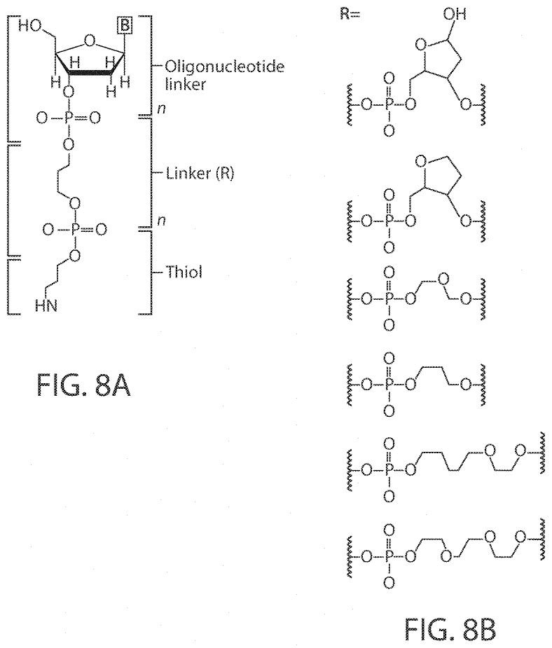

FIG. 8A depicts a polymer synthesized using phosphoramidite methodology to chemically couple the monomers. The linker region incorporates patterns of monomers with varying degrees of immiscible chemical properties (e.g. hydrophobicity, hydrogen/covalent/ionic bonding, etc.). FIG. 8B shows general non-limiting examples of varying chemical functionalities incorporated into the polymer at positions represented by “R.”

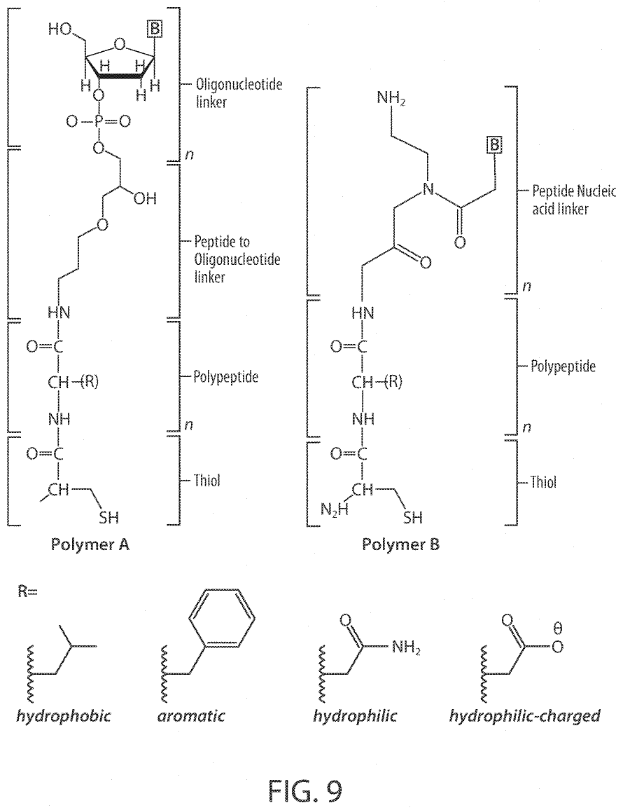

Non-limiting examples of hydrophilic and hydrophobic groups are shown in FIG. 9. The groups may be present within the backbone structure of the polymer and/or as side or pendant groups, in various embodiments. FIG. 9 provides a non-limiting example of a polymer synthesized using amide coupling chemical methodologies standard in peptide synthesis. Amino acid monomers can provide the patterning of chemical functionality useful for chemical interactions between polymers. The amino acid cysteine may provide the thiol moiety for linking the polymer to the nanoparticle. Polymer A in FIG. 9 shows an example of a peptide based polymer with a nucleic acid sequence attached by a peptide to oligonucleotide linker moiety. Polymer B in FIG. 9 incorporates the nitrogenous bases within a peptide nucleic acid based monomer, eliminating the need for a peptide to oligonucleotide linker moiety. Non-limiting examples of varying chemical functionalities incorporated into the polymer at positions represented by “R” are all of the canonical amino acid chemical functionalities (e.g., alanine, arginine, asparagine, aspartic acid, cysteine, glutamine, glutamic acid, glycine, histidine, isoleucine, leucine, lysine, methionine, phenylalanine, proline, serine, threonine, tryptophan, tyrosine, and valine), in addition to non-canonical functionalities ranging in hydrophobicity from hydrophobic hydrocarbons and halogenated compounds to hydrophilic, anionic and cationic chemical functionalities.

Representative examples of hydrophobic functionalities are hydrocarbons in the form of straight, branched, or cyclic structures with potential for varying degrees of unsaturation. Hexyl, 2-methyl-pentyl, trans-2-hexenyl, and cyclohexyl are representative hydrocarbon “R” groups. Aromatic functionalities can represent the “R” group, like phenyl or napthyl groups. Halogenated functionalities like tri-fluoromethyl can be incorporated in the “R” group. Hydrophilic functionalities can be non-ionic or ionic. Representative functionalities including ethers, esters, alcohols, acetals, amines, amides, aldehydes, ketones, nitriles, carboxylic acids, sulfates, sulfonates, phosphates, phosphonates, and nitro groups can be incorporated into the “R” group as ethylene glycol or butanenitrile, for example. Absence of an “R” group may be represented by a hydrogen or unsaturation. These examples represent the types of chemical functionalities useful for chemical interactions between polymers, but are not an inclusive list.

The polymer may include any suitable number of hydrophilic and hydrophobic groups, e.g., to form unique miscibilities suitable for attaching suitable binding partners to a face of a nanoparticle. In some cases, there may be at least 3, at least 4, at least 5, at least 6, at least 7, at least 10, at least 12, at least 15, at least 20, at least 25, at least 30, at least 35, at least 40, at least 45, at least 50, at least 55, at least 60, at least 70, at least 80, at least 90, or at least 100 such groups present. Such numbers may allow for relatively large numbers of unique miscibilities to be generated. For example, in a system comprising a polymer that can include a hydrophilic portion or a hydrophobic portion, 3 monomers would allow 2³=8 possibilities, while 10 monomers would allow 210=10²⁴ possibilities. In such fashion, relatively large numbers of unique patches may be used within a plurality of nanoparticles to build up a nanostructure.

The nanoparticles may be formed into a wide variety of nanostructures that can be used in molecular superstructures such as those described herein, in various embodiments. Any number of nanoparticles may be used to form a nanostructure. For example, 1, 2, 3, 4, 5, 6, 7, 8, 9, or 10 or more, 15 or more, 20 or more, or 25 or more nanoparticles may be present in a nanostructure. If more than one nanostructure is present within a molecular superstructure, the nanostructures may each independently have the same, or different, numbers of nanoparticles.

In one set of embodiments, the nanocubes and/or other nanostructures can be assembled together linearly to form a “nanochain,” e.g., of nanoparticles. The nanochain may be a geometrically straight line of nanoparticles (nanocubes in this example, although other shapes can also be used), e.g., as shown in FIG. 7A, or the nanochain may comprise a topologically linear arrangement of nanoparticles, even if not geometrically straight, e.g., as is shown in FIG. 7B. Thus, for example, one, two, or more “bends” may be present within the nanostructure, e.g., forming L-shapes, zigzags, or the like. In addition, in some cases, branching shapes or other topologically non-linear arrangement of nanoparticles may be used, e.g., as is shown in FIG. 7D.

In some cases, any of the above-described arrangements of nanoparticles may be formed as a planar arrangement, such as is shown in FIG. 7C, e.g., such that there is a single layer of nanoparticles in one dimension. However, in other cases, such as is shown in FIG. 7B, any of the above-described arrangements of nanoparticles may be such that the arrangement is non-planar.

In one set of embodiments, the nanoparticles are positioned within the nanostructure such that the nanostructure has a largest internal dimension (not exiting the nanostructure) of at least about 10 nm, at least about 20 nm, at least about 30 nm, at least about 50 nm, at least about 100 nm, at least about 200 nm, at least about 300 nm, at least about 500 nm, at least about 1 micrometer, at least about 2 micrometers, at least about 3 micrometers, at least about 5 micrometers, at least about 10 micrometers, at least about 20 micrometers, at least about 30 micrometers, at least about 50 micrometers, at least about 100 micrometers, at least about 200 micrometers, at least about 300 micrometers, at least about 500 micrometers, at least about 1000 micrometers, etc. In some cases, the largest internal dimension may be less than about 1000 micrometers, less than about 500 micrometers, less than about 300 micrometers, less than about 200 micrometers, less than about 100 micrometers, less than about 50 micrometers, less than about 30 micrometers, less than about 20 micrometers, less than about 10 micrometers, less than about 5 micrometers, less than about 3 micrometers, less than about 2 micrometers, less than about 1 micrometers, less than about 500 nm, less than about 300 nm, less than about 200 nm, less than about 100 nm, less than about 50 nm, less than about 30 nm, less than about 20 nm, less than about 10 nm, etc. The largest internal dimension may also be a combination of any of these, e.g., between 500 nm and 1000 nm.

In some embodiments, the nanoparticles are positioned within the nanostructure such that the nanostructure has a maximum dimension (the maximum possible distance that the nanostructure can be positioned so as to separate two imaginary parallel planes) of at least about 10 nm, at least about 20 nm, at least about 30 nm, at least about 50 nm, at least about 100 nm, at least about 200 nm, at least about 300 nm, at least about 500 nm, at least about 1 micrometer, at least about 2 micrometers, at least about 3 micrometers, at least about 5 micrometers, at least about 10 micrometers, at least about 20 micrometers, at least about 30 micrometers, at least about 50 micrometers, at least about 100 micrometers, at least about 200 micrometers, at least about 300 micrometers, at least about 500 micrometers, at least about 1000 micrometers, etc. In some cases, the maximum dimension may be less than about 1000 micrometers, less than about 500 micrometers, less than about 300 micrometers, less than about 200 micrometers, less than about 100 micrometers, less than about 50 micrometers, less than about 30 micrometers, less than about 20 micrometers, less than about 10 micrometers, less than about 5 micrometers, less than about 3 micrometers, less than about 2 micrometers, less than about 1 micrometers, less than about 500 nm, less than about 300 nm, less than about 200 nm, less than about 100 nm, less than about 50 nm, less than about 30 nm, less than about 20 nm, less than about 10 nm, etc. The largest maximum dimension may also be a combination of any of these, e.g., between 500 nm and 1000 nm.

In some embodiments, the nanoparticles are positioned within the nanostructure such that the nanostructure has a minimum dimension (the minimum possible distance that the nanostructure can be positioned so as to separate two imaginary parallel planes) of at least about 10 nm, at least about 20 nm, at least about 30 nm, at least about 50 nm, at least about 100 nm, at least about 200 nm, at least about 300 nm, at least about 500 nm, at least about 1 micrometer, at least about 2 micrometers, at least about 3 micrometers, at least about 5 micrometers, at least about 10 micrometers, at least about 20 micrometers, at least about 30 micrometers, at least about 50 micrometers, at least about 100 micrometers, at least about 200 micrometers, at least about 300 micrometers, at least about 500 micrometers, at least about 1000 micrometers, etc. In some cases, the minimum dimension may be less than about 1000 micrometers, less than about 500 micrometers, less than about 300 micrometers, less than about 200 micrometers, less than about 100 micrometers, less than about 50 micrometers, less than about 30 micrometers, less than about 20 micrometers, less than about 10 micrometers, less than about 5 micrometers, less than about 3 micrometers, less than about 2 micrometers, less than about 1 micrometers, less than about 500 nm, less than about 300 nm, less than about 200 nm, less than about 100 nm, less than about 50 nm, less than about 30 nm, less than about 20 nm, less than about 10 nm, etc. The largest minimum dimension may also be a combination of any of these, e.g., between 500 nm and 1000 nm.

In some cases, the nanostructures may be formed using self-assembly or other techniques. For example, for nanoparticles such as nanocubes having faces featuring selectively binding patches may be combined, e.g., in solution or suspension, with other nanoparticles having complementary patches on one or more of their faces, to produce nanostructures. In some cases, this process may be facilitated through stirring or other mechanical actions.

The nanoparticles may be able to self-assemble to produce one or more specific, predefined, nanostructures, e.g., of varying geometrical shapes, according to certain embodiments. In contrast, in many prior art techniques, self-assembly of nanoparticles results in uncontrolled aggregation of nanoparticles into a non-predetermined or uncontrollable shape, which, aside from trivial modifications such as nanoparticle size and linker length, does not confer a significant degree of control of the final nanostructure that is formed.

In one set of embodiments, the nanostructure may comprise at least 2, at least 3, at least 5, at least 8, at least 10, at least 15, at least 20, at least 25, at least 30, at least 40, at least 50, at least 60, at least 70, at least 80, at least 90, at least 100, at least 150, at least 200, at least 300, at least 400, at least 500, at least 750, at least 1000, at least 3000, at least 5000, or at least 10,000 nanoparticles. In some cases, each of the nanoparticles have unique arrangements of patches. In other cases, however, some of the nanoparticles within the nanostructure may be identical to each other.

Dimer aggregates may be formed as the complementary patches bind together. Larger aggregates comprised of more nanoparticles can also be formed in various embodiments, representing a general method for synthesizing arbitrarily shaped three-dimensional nanostructure, regardless of how anisotropic or complex the target nanostructure may be.

In some cases, the nanoparticles may be considered to represent a “pixel” (e.g. a nanocube pixel) within a larger nanostructure, in two or three dimensions. The patches may be selected so as to determine where each “pixel” will appear within the nanostructure. By controlling the location of patches on individual nanoparticles, complex nanostructures may be obtained with almost any suitable shape. In some cases, the synthesis may involve only one type of building block (e.g., only one type of nanoparticle), which may reduce the complexity of the assembly process, while simultaneously expanding the complexity of the nanostructures that can be built. As such, this may reduce the number of synthetic techniques one needs to assemble a variety of different shapes, and could be adopted as a standardized technique to assemble large classes of nanostructures. However, it should be understood that in other embodiments, more than one type of nanoparticle may be present, e.g., having different shapes, sizes, materials, etc., as discussed herein.

In one embodiment, nanoparticles are directly connected to each other, e.g., in a face-to-face orientation, to form a nanostructure. It should be understood that the orientation may be exact, or in some cases, the alignment of nanoparticles may be off-center. As a specific non-limiting example, DNA ligands covering a face of one nanoparticle may hybridize to DNA ligands on the face of another nanoparticle. By preparing the nanoparticle faces with known DNA sequences in advance, as discussed herein, and then combining the nanoparticle in solution or suspension, aggregates may form as the DNA-coated faces bind to other faces containing the complementary strand. If the connections are unique, then only a specific superstructure may form, e.g., one that is programmable or predetermined.

However, while linker DNA is not necessarily used in all embodiments, linker DNA can be used in some cases. For example, when forming large structures, the kinetics may result in higher yields if the hybridization reactions proceed in a certain order. Adding linker strands in progression, e.g., to the solution or suspension containing nanoparticles, may control the order in which nanoparticles bind together to form the larger nanostructure.

Yet another embodiment uses the addition of an ssDNA (or other suitable nucleic acid) as a linker to initialize the hybridization of multiple nanoparticles. To build nanostructures from the nanoparticles, one may combine the nanoparticles to be linked in solution or suspension along with appropriate ssDNA linker strands. The addition of a linker may allow, in some cases, the order in which nanoparticles bind to each other to be specified. In some cases, for example, this may increase the yield of the nanostructures by avoiding kinetic traps, e.g., where the correct nanostructure is not able to be formed.

Nanoparticles may be readily obtained commercially, and/or synthesized as discussed herein. In one embodiment, the nanoparticles may be nanocubes. A nanocube typically is substantially cube-shaped, although in reality, such nanocubes are not expected to be mathematically-perfect cubes. In practice, the dimensions and/or angles of such nanocubes may accordingly vary somewhat from the ideal mathematical cube. For instance, the nanocubes may have a height, length, or width that varies less than 20 nm, less than 15 nm, less than 10 nm, or less than 5 nm of the other dimensions, and/or the angles defining the nanocube may not be precisely 90°, but may be between 80° and 100°, or between 85° and 95°, etc.

In addition to nanocubes, the nanoparticles may have other shapes as well, such as cylinders, plates, prisms, rectangular solids (which may or may not have a square face, and which may be orthogonal or may be skewed or non-orthogonal in 2 or 3 dimensions), or other platonic solids (e.g., tetrahedron, octahedron, dodecahedron, or icosahedron). Thus, in further embodiments, a variety of other faceted nanoparticle shapes can be synthesized, including tetrahedrons, octahedrons, and icosahedrons, to name a few. In some cases, the nanoparticles have a shape such that they may be stacked together without gaps, e.g., such as cubes, rhombic dodecahedrons, truncated octahedrons, tetrahedron/octahedron honeycombs, or other 3-dimensional tessellation shapes. The nanoparticles may also have semiregular or irregular shapes in some embodiments. In certain embodiments, the outer surface of nanoparticle is defined by substantially flat planar surfaces, e.g., as in a polyhedron. There may be any suitable number of flat surfaces defining the nanoparticle, e.g., 4, 5, 6, 7, 8, 9, 10, 11, 12, 13, 14, 15, 16, etc. The faces may independently be of the same or different shapes and/or sizes, and may be regular or irregular. In some cases, the nanoparticles have at least one pair of opposed sides that are parallel to each other, and in certain cases, the nanoparticles may have two, three, or more pairs of opposed sides that are parallel to each other.

A nanocube or other nanoparticle typically has a largest internal dimension of less than about 1 micrometer, e.g., such that it is measured on the order of nanometers. For example, in some cases, the nanoparticle may have a largest internal dimension of less than about 100 micrometers, less than about 50 micrometers, less than about 30 micrometers, less than about 20 micrometers, less than about 10 micrometers, less than about 5 micrometers, less than about 3 micrometers, less than about 2 micrometers, less than about 1 micrometer, less than about 900 nm, less than about 800 nm, less than about 700 nm, less than about 600 nm, less than about 500 nm, less than about 400 nm, less than about 300 nm, less than about 200 nm, less than about 100 nm, less than about 90 nm, less than about 80 nm, less than about 70 nm, less than about 60 nm, less than about 50 nm, less than about 40 nm, less than about 30 nm, less than about 20 nm, or less than about 10 nm. In some cases, the nanocube or other nanoparticle may have a largest internal dimension of at least about 10 nm, at least about 20 nm, at least about 30 nm, at least about 50 nm, at least about 100 nm, at least about 200 nm, at least about 300 nm, at least about 400 nm, at least about 500 nm, at least about 600 nm, at least about 700 nm, at least about 800 nm, at least about 900 nm, at least about 1 micrometer, at least about 2 micrometers, at least about 3 micrometers, at least about 5 micrometers, at least about 10 micrometers, etc. Combinations of any of these are also possible, e.g., a nanocube or other nanoparticle may have a largest internal dimension of between 1 nm and 1000 nm.

The nanoparticles may be formed from any suitable material. Examples of nanoparticle compositions useful in various embodiments of the invention include metals (e.g. gold, silver, platinum, copper, and iron, etc.), semiconductors (e.g. silicon, silicon, copper selenide, copper oxide, cesium oxide, etc.), magnetic materials (e.g., iron oxide), or the like. Combinations of these are also possible, e.g., gold-silver nanoparticles, gold-copper nanoparticles, etc. In some cases, the nanoparticle comprises an alloy of 2, 3, or more metals. Methods of making nanoparticles with different compositions and/or geometries are known in the art.

For example, in one set of embodiments, nanoparticles may be created using polyol-mediated synthesis. Polyol mediated synthesis of nanoparticles may be initiated in some cases by reduction of a metal salt into a metal ion at high temperature. A capping agent may interact with a nanoparticle surface to influence the nanoparticle size and shape. In various embodiments, ethylene glycol, a polyol, can act as both the reducing agent and the capping agent, in addition to capping agents (e.g. polyvinylpyrrolidone and cetyltrimethylammonium bromide (CTAB)) and reducing agents (e.g. sodium hydrosulfide and ascorbic acid).

In some embodiments, the composition of the nanoparticle can be determined by the identity of the metal salt used. For example, silver nitrate can be used for synthesis of silver nanoparticles and gold chloride can be used for synthesis of gold nanoparticles. Other metal nanoparticles such as those discussed herein can be prepared using corresponding metal salts, e.g., metal chlorides or metal nitrates.

The size and shape of the nanoparticle can be controlled in various embodiments by controlling reaction conditions like the reaction time, identity of the reaction components (e.g. capping and reducing agents), and/or the concentration of components in the reaction. For example, the size of the nanoparticles can be controlled by quenching a synthesis reaction at a desired time. In some embodiments, the shape of the nanoparticles may be controlled by controlling the concentrations of capping agents and/or reducing agents. For example, gold nanocubes can be formed using low CTAB and high ascorbic acid concentrations, whereas high CTAB and low ascorbic acid concentrations may favor formation of octahedral shapes in certain embodiments.

In one set of exemplary embodiments, gold nanoparticles are utilized. For example, gold, in the form of a salt, may be dissolved in solvent and reduced by a reducing agent. The size and morphology of the gold nanoparticles may be controlled by the addition of capping agents to the reaction. The capping agent can be attached to the surface of the gold nanoparticle, kinetically or thermodynamically inhibiting additional atoms from joining the crystal. Gold nanoparticles can be purified by a variety of methodologies, including centrifugation, column chromatography, and gel electrophoresis.

In some cases, more than one nanoparticle may be present, including any combination of any of those discussed herein. For instance, if more than one type of nanoparticle is present, the nanoparticles may independently differ on the basis of shape, size, material, or the like, and/or combinations thereof. For example, there may be two, three, or more sizes of nanocubes present, and/or there may be a variety of different shapes of nanoparticles present (e.g., nanotetrahedrons and/or nanoctahedrons), and/or there may be a variety of nanoparticles comprising different materials that are present.

The nanoparticles that are present may have a narrow size distribution in some embodiments. For instance, the nanoparticles may have a distribution such that less than about 30%, less than about 20%, less than about 10%, less than about 5% of the nanoparticles have a largest internal dimension that is greater than 120% or less than 80%, or greater than 110% or less than 90%, of the average largest internal dimension of all of the nanoparticles.

As a specific non-limiting example, in one set of embodiments, silver nanocubes may be used with an edge length of greater than 100 nm as nanoparticles. In some cases, all six faces of the nanocubes can be coated with single-stranded oligonucleotides in a manner that each face is homogeneously coated with many of the same type of oligonucleotide. Oligonucleotide sequences may be patterned on the faces of the nanocube such that two faces on opposite ends are coated with oligonucleotides of different sequences than those coating the four circumferential faces of the nanocube. A terminal thiol moiety, present on the 3′ and/or 5′ end of the single-stranded oligonucleotide, can be used to provide direct attachment of the oligonucleotides to the nanocube surface. Extending from the thiol moiety, a hexaethylene glycol polymer is included, followed by a sequence of 20 nucleotides.

In some embodiments, an oligonucleotide linker may be included with the oligonucleotide-coated nanocubes that is complementary to the oligonucleotides patterned on opposite faces of the nanocube. This oligonucleotide linker may hybridize to the oligonucleotides on the nanocube surface, causing one face of a nanocube to attach to another nanocube. The attachment of the oligonucleotide linker to the oligonucleotides on the faces of the nanocubes may be used to join two or more nanocubes into a nanochain, e.g., in a manner akin to polymerization. In some instances, it may be advantageous to form multiple species of polymeric nanoparticle chains with distinct oligonucleotide sequences on the nanoparticle surface. For example, in one set of embodiments, two nanoparticle chains with different circumferential oligonucleotides, which are complimentary to different regions of a target molecule of interest, may be used.

Certain aspects of the present invention are generally directed to molecular superstructures that are formed as discussed herein. In some cases, for example, suitable nanostructures may be induced to assemble together to form a superstructure, for example, spontaneously (e.g., self-assembly), and/or through the addition of other agents, such as linker, to cause assembly to occur, e.g., in the presence of one or more target molecules. In some cases, a single molecular superstructure is assembled from the nanostructures; in other cases, however, more than one such molecular superstructure may be assembled. Thus, in some embodiments, a plurality of superstructures are formed. In some cases, at least 20%, at least 30%, at least 40%, at least 50%, at least 60%, at least 70%, at least 80%, at least 90%, at least 95%, or substantially all of the molecular superstructures that are formed may share essentially identical configurations of nanostructures forming those superstructures. In one set of embodiments, the superstructures are formed in a solution or suspension comprising nanostructures. In some cases, the superstructures that are formed are solid or stably formed from nanostructures, e.g., the superstructure has a well-defined shape or structure under ambient conditions (e.g., at room temperature and pressure). In some embodiments, the superstructure may be stable or have a solid form even when contained within solution or suspension, e.g., such that the superstructure does not typically dissociate or “fall apart” when left undisturbed under room temperature and ambient pressure, even in the presence of normal fluidic flow within the solution or suspension. The shape of the superstructure can be programmed or predetermined in certain instances, e.g., as discussed herein, for example, in the presence (or absence) of target molecules.

Thus, in certain embodiments, one or more nanostructures are assembled using target molecules to form a molecule superstructure. There may be, for example, 1, 2, 3, 4, 5, 6, 7, 8, 9, or 10 or more, 15 or more, 20 or more, or 25 or more target molecules present within a molecular superstructure.

The target molecule of interest may be any suitable target that can bind to or otherwise interact to form part of the molecule superstructure. For example, the target molecule may bind to or otherwise interact with a nanoparticle of a nanochain, which may form part of the superstructure. Such binding may occur at one, two, there, four, or more points of the target molecule, and the bindings may occur to the same or different nanoparticles and/or nanochains. The binding may occur through a variety of interactions, such as via hydrogen bonds, van der Waals forces, hydrophobic interactions, covalent coupling, or the like. The binding may be specific or non-specific, in various embodiments. For instance, in a binding interaction, there may be only one possible complementary binding partner (for example, complementary nucleic acid strands), or there may be a variety of possible binding partners.

In some embodiments, the target molecule may be a polymer, such as a protein, a carbohydrate, a nucleic acid, or the like. In some cases, the polymer may be a synthetic or human-made polymer. If a protein, the protein in some cases may be denatured, e.g., to facilitate interaction between the proteins and suitable binding sites, e.g., in a nanochain. However, the protein may not necessarily be denatured.

In some cases, the target molecule may have a length of at least 5 nm, at least 10 nm, at least 15 nm, at least 20 nm, at least 25 nm, at least 30 nm, at least 40 nm, at least 50 nm, at least 60 nm, at least 70 nm, at least 80 nm, at least 90 nm, at least 100 nm, at least 200 nm, at least 300 nm, at least 500 nm, or at least 1,000 nm. In addition, in some embodiments, the target molecule may be a polymer having at least 2, at least 3, at least 5, at least 10, at least 15, at least 20, at least 30, at least 40, at least 50, at least 100, at least 200, at least 300, at least 500, or at least 1,000 subunits.

The target molecule may be a nucleic acid in some embodiments. For example, the target may include DNA and/or RNA. The target may have any suitable length, for example, at least 100 nt, at least 300 nt, at least 500 nt, at least 1,000 nt, at least 3,000 nt, at least 5,000 nt, at least 10,000 nt, at least 30,000 nt, at least 50,000 nt, at least 100,000 nt, at least 300,000 nt, at least 500,000 nt, at least 1,000,000 nt, etc. For example, in one set of embodiments, the target nucleic acid may comprise genomic DNA.

As a non-limiting example, in one set of embodiments, the target molecule of interest may be genomic DNA with a particular nucleotide sequence. The sample material containing the target nucleotide sequence may be introduced to nanostructures such as those discussed herein, for example, oligonucleotide-coated nanocubes and linker oligonucleotides. In some cases, the nanostructures may be heated to an elevated temperature, e.g., to help denature the double-stranded genomic DNA sufficiently enough to cause the genetic region of interest to become single-stranded. The components can be introduced together or in any order. Additives that aid in denaturation of the genomic DNA may be included in some embodiments. For example, several oligonucleotides can be included that are complementary to the genomic DNA sequence on the strand opposing target strand. These oligonucleotides may aid in the availability of single-stranded genomic DNA. In some cases, two different oligonucleotides may be included along with the mixture of nanocubes that hybridize to the nanocube faces of oriented 180 degrees to each other, e.g., forming two polymeric nanoparticle chains with distinct oligonucleotide sequences on the nanoparticle surface.

Upon exposure of single-stranded genomic DNA, oligonucleotides on the nanocube surface may hybridize to the complementary DNA genomic sequence. For example, in a mixture that contains nanocubes whose faces, not including the two faces used for assembly of the polymer chain, are coated in oligonucleotides that are complementary to two different sites on one strand of the genomic DNA, polymeric chains of nanocubes may be able to assemble, for example, into ordered arrays. In some cases, the ordered arrays may have a spacing that is proportional to the number of nucleotides located between the two different binding sites in the genomic DNA. For example, two binding sites in the genomic DNA that are 1,300 nucleotides apart may account for a dynamic spacing between the polymeric nanocube chains on the order of 450 nm.

Genetic material may or may not require processing prior to mixing as discussed above. Examples of processing may include, but are not limited to, dissolving genetic material in an aqueous solution, concentrating, or extracting genetic material from unwanted components in the sample matrix, or may include addition to or alone, the enzymatic amplification of the target nucleotide sequence, or the fragmentation of the nucleotide sequence mechanically or enzymatically.

Thus, in accordance with certain embodiments, a first nanostructure may bind to a first location of a target molecule, such as a nucleic acid, while a second nanostructure may bind to a second location of the target molecule. One or both binding locations may be specific. For example, the first and second sites within a target molecule may be separated by at least about 10 nm, at least about 20 nm, at least about 30 nm, at least about 50 nm, at least about 100 nm, at least about 200 nm, at least about 300 nm, at least about 400 nm, at least about 500 nm, at least about 600 nm, at least about 700 nm, at least about 800 nm, at least about 900 nm, at least about 1 micrometer, at least about 2 micrometers, at least about 3 micrometers, at least about 5 micrometers, at least about 10 micrometers, etc. In some cases, the separation between the first and second sites may be less than about 10 micrometers, less than about 5 micrometers, less than about 3 micrometers, less than about 2 micrometers, less than about 1 micrometers, less than about 900 nm, less than about 800 nm, less than about 700 nm, less than about 600 nm, less than about 500 nm, less than about 400 nm, less than about 300 nm, less than about 200 nm, less than about 100 nm, less than about 50 nm, less than about 30 nm, less than about 20 nm, less than about 10 nm, etc. In addition, in some cases, the separation may be such that the molecular superstructure is able to diffract light, such as visible light (400 nm to 700 nm), infrared light (700 nm to 1 mm), and/or ultraviolet light (10 nm to 400 nm). In addition, if the target molecule is a nucleic acid, then the first and second sites may be separated by at least 100 nt, at least 300 nt, at least 500 nt, at least 1,000 nt, at least 3,000 nt, at least 5,000 nt, at least 10,000 nt, at least 30,000 nt, at least 50,000 nt, at least 100,000 nt, at least 300,000 nt, at least 500,000 nt, at least 1,000,000 nt, etc.

If more than one target molecule is present, e.g., within a molecular superstructure, then each of the target molecules may independently have the same or different separations of binding sites, e.g., to the same or different nanostructures.

In one set of embodiments, more than one target molecule of interest may be determined in a sample. The target molecules may bind to the same or different molecular superstructures for detection. The target molecules may be determined independently of each other, or in some cases, the target molecules may be determined together.

For example, in one set of embodiments, a first target molecule and a second, different target molecule may be needed in order for a molecular superstructure to form. Thus, the presence of one target molecule and not the other may not be sufficient to form the final molecular superstructure, and/or the molecule structure cannot be fully assembled, e.g., it may partially assemble, or assemble into a different configuration (e.g., one that is not determinable, or can be determined as being incorrectly assembled).

As another example, a first target molecule may be used to form a first molecular superstructure, while a second target molecule may be used to form a second molecular superstructure distinguishable from the first molecular superstructure in some fashion. For example, the two molecular superstructures may produce different diffraction or scattering patterns, or other differences that can be determined, e.g., as is discussed herein. Thus, as a non-limiting example, the presence of a first diffraction pattern can be used to determine the first molecular superstructure while the presence of a second diffraction pattern can be used to determine the second molecular superstructure

In some embodiments, for example, multiple nucleotide sequences in a sample can be detected simultaneously using hybridization or binding sites of the nanostructures to have a distinct spacing, for example, depending on properties such as different target nucleotide sequences. The scattering angle of the incident light may be a function of the spacing between the nanostructures within the molecular superstructures. This spacing may be determined, for example, by the number of nucleotides between the binding sites on the target molecule, e.g., as is discussed herein. This spacing can then be determined, for example, using various determination methods discussed herein, for example, by determining diffraction angles, scattering, or the like.

Thus, the presence of one or more target molecules in a sample can be indicated by the presence or absence of one or more of the predicted spacings between nanostructures assembled into a molecular superstructure. In some cases, a variety of techniques, including microscopy or detection of light scattered at the predicted angle, may be used to determine such spacing.

In some embodiments, as mentioned, multiple target molecules may be determined simultaneously. As a non-limiting example, two or more sample wells that have access to the same sample matrix may be used. By flow of one sample matrix into multiple wells or chambers containing suitable nanostructures, multiple samples can be determined in parallel. Many sample wells or chambers can be determined simultaneously and/or in an automated fashion, etc., while the sample wells or chamber may be analyzed, e.g., independently or in parallel, for the presence of nanostructures assembled into molecular superstructures.

In one set of embodiments, one or more of the nanostructures may be immobilized relative to a surface, e.g., directly or indirectly. For example, in some cases, a linker or a tether may be used to immobilize a nanostructure relative to a surface. In some embodiments, by immobilizing nanostructures relative to a surface, a target molecule can be removed from the nanostructures, e.g., without removing the nanostructures themselves. Thus, for example, a first sample may be determined, then removed and replaced with a second sample. Accordingly, in some embodiments, the nanostructures may be used multiple times, e.g., for determining target molecules within various samples. In addition, in some embodiments, multiple nanostructures may be immobilized relative to a surface, e.g., using independent linkers or tethers, which may be sufficiently flexible such that, in the presence of a suitable target molecule, the multiple nanostructures are able to form a molecular superstructure, such as is discussed herein.

A variety of methods may be used to tether or link a nanostructure to a surface. In some cases, one end of the tether may be attached or bonded to a nanostructure (e.g., using covalent bonding), while the other end of the tether may be attached or bonded to a surface. Non-limiting examples of chemical moieties that may be used include polyethylene glycol, amide-linked polymers such as nylon, polypeptides, nucleic acids such as DNA or RNA, or the like.

A variety of chemical moieties may be used on the terminal end of the tether, and these may depend, at least in part, on the chemical composition of the surface. For example, silicon, glass, and mica surfaces can functionalized with an aminosilane monolayer, providing a free amine for chemical coupling to the tether molecule. In another embodiment, carbodiimide catalyzed coupling of a carboxylic acid to the free amine can bind the tether to the surface. In some cases, a thiol moiety can be used to the tether to a gold or silver surface.

In some aspects, the separation of binding sites on a target molecule, such as a nucleic acid, may allow the nanostructures to be substantially regularly spaced within the molecular superstructure, e.g., upon interaction or binding with the target molecule. For example, the positioning of the nanostructures within the molecular superstructure may allow the superstructure to act as a diffraction grating, or otherwise scatter or diffract light, which can be determined in some fashion. Accordingly, a variety of techniques can be used to determine the superstructure, e.g., qualitatively and/or quantitatively.

For example, in one set of embodiments, light may be applied to at least a portion of the sample to determine the molecular superstructures. The presence of molecular superstructures may alter the incident light, for example, by reflecting, refracting, diffracting, scattering, etc. the incident light. For instance, the molecular superstructures may cause the incident light to scatter or diffract, for example, by acting as a diffraction grating (e.g., as formed from nanostructures within the molecular superstructure, e.g., substantially regularly-spaced). In contrast, if the molecular superstructures are absent (e.g., due to a lack of target molecules), then no scattering or diffraction may occur. In this way, the presence or absence of target molecules may be determined. In addition, in some cases, this may be quantified to determine the target molecules quantitatively; for example, the amount of scattering or the intensity of the diffracted light may be used to quantitatively determine the target molecules.

Accordingly, the light that is applied may be any suitable light able to interact with molecular superstructures. In some cases, the light has a frequency substantially equal, or that includes, the dynamic spacings of nanostructures within the molecular superstructure. For example, the light that is applied may be visible light (400 nm to 700 nm), infrared light (700 nm to 1 mm), ultraviolet light (10 nm to 400 nm), or combinations thereof. In some cases, the light is relatively monochromatic or coherent (e.g., laser light), although in some embodiments, light of multiple frequencies (for example, white light) may be used.

In some cases, the light that is applied has a wavelength of at least about 10 nm, at least about 20 nm, at least about 30 nm, at least about 50 nm, at least about 100 nm, at least about 200 nm, at least about 300 nm, at least about 400 nm, at least about 500 nm, at least about 600 nm, at least about 700 nm, at least about 800 nm, at least about 900 nm, at least about 1 micrometer, at least about 2 micrometers, at least about 3 micrometers, at least about 5 micrometers, at least about 10 micrometers, etc. In some cases, the wavelength may be less than about 10 micrometers, less than about 5 micrometers, less than about 3 micrometers, less than about 2 micrometers, less than about 1 micrometers, less than about 900 nm, less than about 800 nm, less than about 700 nm, less than about 600 nm, less than about 500 nm, less than about 400 nm, less than about 300 nm, less than about 200 nm, less than about 100 nm, less than about 50 nm, less than about 30 nm, less than about 20 nm, less than about 10 nm, etc. The light may also include combinations of any of these wavelengths.

As a non-limiting example, a molecular superstructure may be determined by observing unique diffraction patterns created by the interaction with incident electromagnetic waves. Diffraction patterns may appear when waves pass through or reflect off a superstructure. A monochromatic light source may be used in some embodiments. Such a monochromatic light source may provide a well-defined diffraction pattern, as the scattering angle of the diffracted light is a function of the wavelength of light and the spacing between nanostructures in the molecular superstructure. The spacing of the nanostructures may in some cases be greater than or equal to the wavelength of light to efficiently induce scattering of incident light. As a non-limiting example, in one embodiment, a diode laser emitting a wavelength of light at 405 nm may be used to provide a diffraction pattern with a spacing between polymeric nanocube chains on the order of 450 nm.

In some embodiments, determination of nanostructures using light diffraction (or other interactions) can be performed by techniques such as visual identification of light scattering or by electronic detection. In one embodiment, a linear CCD array may be used to determine the scattering of light. In other embodiments, other detection techniques may be used to determine such light, such as photodiodes. A variety of suitable detectors may also be used, many of which are commercially available. Non-limiting examples include cameras such as CCD cameras, photodiodes, photodiode arrays, or the like. In addition, various optical components may be used in some embodiments to assist in directing the light towards the detector, for example, lenses, mirrors, beam splitters, filters, slits, windows, prisms, diffraction gratings, optical fibers, etc.

However, it should be understood that the invention is not limited to only monochromatic light. A variety of other techniques can be used in various embodiments to determine molecular superstructures. Even the unaided eye may be used to determine molecular superstructures in certain embodiments. For instance, samples containing molecular superstructures may appear to be “cloudy,” if the superstructures are sufficiently large (for example, molecular superstructures that have assembled in the presence of target molecules), while samples lacking such molecular superstructures (for example, lacking appropriate target molecules) may appear clear (for instance, if the nanostructures are sized to be optically smaller than the wavelength of visible light).

Other methods of determination include optical or microscopic techniques. For example, microscopy techniques such as visible microscopy, electron microscopy, or atomic force microscopy may be used. In some cases, a sample may be prepared, for example, on a surface, which can then be studied using such microscopy techniques to determine the molecular superstructures. In some embodiments, detection of substantially regularly-spaced nanostructures within a sample may be indicative of target molecules, while the lack of such substantially regularly-spaced nanostructures may indicate a lack of target molecules. In addition, in some cases, the amount or concentration of target molecules may be quantified, for example, by determining the degree that the nanostructures are substantially regularly-spaced within the sample, as determined using such techniques.

As a non-limiting example, in one set of embodiments, determination of a molecular superstructure by microscopy of the sample may be performed in solution or suspension, or in dried form. In some cases, randomly-oriented nanostructures may be used to indicate a negative result for the presence of a target molecule (e.g., a genomic DNA sequence), whereas, a positive result may be indicated by the observation of regularly-spaced nanostructures. Using microscopy or other techniques, the determination of regularly-spaced nanostructures can be achieved, for example, by visual identification or a computer. For example, a computer may be used to process a digitized image algorithmically to determine such regularly-spaced nanostructures.

As mentioned, in certain aspects of the invention, more than one sample may be determined for target molecules. Thus, certain embodiments of the invention may be reused to determine multiple samples. For example, in some embodiments, the association of target molecules and nanostructures may be substantially reversible, and the nanostructures may be reused between different reactions to determine different samples or target molecules. In some cases, nanostructures such as those described herein may be retained, e.g., within a sample chamber, using techniques such as chemical binding (e.g., via a tether or a linker) or physical retention (e.g., if the nanostructures are of a size too large to exit the sample chamber), etc.

As a non-limiting example, hybridization of complementary nucleotide sequences is a reversible process. Elevated temperatures may disfavor hybridization of complementary nucleotide sequences. Chemical denaturants that interfere with the base pairing interactions between strands of nucleotides, such as formimide, may be used to disfavor hybridization of complementary oligonucleotide sequences, and/or solutions with low ionic strength or a pH that varies from physiological conditions. By subjecting a molecular superstructure assembled with nucleotides to conditions that favor denaturation of complementary nucleotide sequences, the molecular superstructure may be dissembled. Thus, for example, target nucleotide sequences introduced with the sample matrix can be separated from nanoparticles or nanostructures for reuse. As mentioned, in some embodiments, one or more of the nanostructures can be tethered to a surface, e.g., using a linker. A variety of different chemical moieties may be used as tethers, such as polyethylene glycol, nylon, polypeptides, or other moieties such as those described herein.

In some embodiments, one or more nanostructures may be reused in more than one assay for detection of nucleic acid sequences or other suitable target molecules. In some cases, a microfluidic device can provide the separation of such nanostructures from target nucleotide sequences present in a sample by using fluid paths sized to be smaller than the nanostructures. This may be achieved in a variety of ways, such as by using microfluidic channels smaller than the nanostructures, using a membrane having an average pore size smaller than the nanostructures, flowing fluid through a tortuous pathway such that the nanostructures are unable to exit, or the like.

Thus, as a non-limiting example, in some cases, sample suspected of containing the target molecules may flow into a sample chamber containing the nanostructures, and the conditions are provided for assembly of a molecular superstructure if the target molecules are present. Following analysis for the presence of the molecular superstructure, the conditions are changed to disfavor assembly, (for example, by disfavoring the hybridization of complementary nucleotide sequences). The components other than the nanostructures may then be removed. For example, a flow-through the sample chamber may be provided to remove components other than the nanostructures, which may be unable to exit due to their larger size or inflexibility. Thus, another sample can be introduced to the sample chamber and conditions established to assemble molecular superstructures, e.g., if target molecules are present.

International Patent Application No. PCT/US2016/043303, filed Jul. 21, 2016, entitled “Programmable, Self-assembling Patched Nanoparticles, and Associated Devices, Systems, and Methods,” by Santos and Lyons, published as WO 2017/015444 on Jan. 26, 2017, is incorporated herein by reference in its entirety. In addition, incorporated herein by reference in its entirety is U.S. Provisional Patent Application Ser. No. 62/584,286, filed Nov. 10, 2017, entitled “Molecular Detection via Programmable Self-Assembly,” by Lyons and Santos.

The following examples are intended to illustrate certain embodiments of the present invention, but do not exemplify the full scope of the invention.

### Example 1

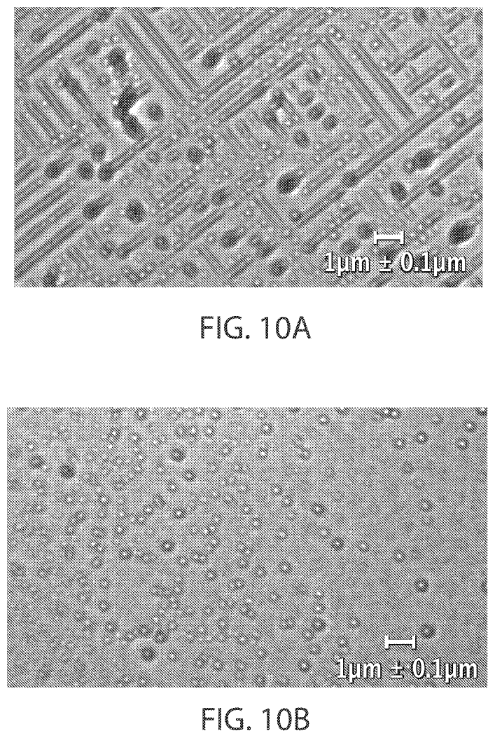

In this example, the detection of tubulin beta-6 chain (TUBB6) gene fragment from maize was accomplished by using silver nanocubes, of 150 nm edge length, with all six faces coated in thiol-linked oligonucleotides. See FIG. 10.

Two nanocube faces were coated with 15-nucleotide single-stranded oligonucleotides terminated with a hexaethylene glycol spacer (6Sp) and a thiol on either the 5′ or 3′ position. Specifically, each of the nanocube faces coated by these two oligonucleotides was oriented on polar opposite faces of the nanocube with 5′-thiol-6Sp-CTCCCTAATAACAATT (SEQ ID NO: 1) and 5′-TTATAACTATTCCTA-6Sp-thiol (SEQ ID NO: 2). These two oligonucleotides were complementary to a 30-nucleotide single-stranded oligonucleotide that links the one nanocube to another, forming a polymeric chain, 5′-TAGGAATAGTTATAAATTGTTATTAGGGAG (SEQ ID NO: 3).

Two different sets of polymeric chains are used in this gene detection method, the second set of polymeric nanocube chains is formed from nanocubes coated with 5′-thiol-6Sp-GTGAGTATTCGTATG (SEQ ID NO: 4) and 5′-GTATCTGATGTGACA-6Sp-thiol (SEQ ID NO: 5) on polar opposite faces and linked into a chain by 5′-TGTCACATCAGATACCATACGATTACTCAG (SEQ ID NO: 6). The remaining four faces of the nanocubes, in an equatorial pattern, were coated with an oligonucleotide that is complementary to a sequence in the target gene, TUBB6 in this example. 5′-thiol-Sp6-CGCTGTTCTCATGGA (SEQ ID NO: 7) was applied to the four remaining faces of one set of nanocubes, while 5′-TGGGATGCCAAGAAC-Sp6-thiol (SEQ ID NO: 8) was applied to the other set of nanocubes. These two sequences were chosen to be ˜1.4 kb apart in the TUBB6 gene, providing roughly 480 nm of linear distance between the two sequences. The number of nucleotides between the two binding sequences in the target genomic DNA sequence determines the average spacing between any two polymeric nanocube chains in the assembled superstructure of parallel nanocube chains.

The two sets of polymeric chains, formed by the presence of the linker oligonucleotides, hybridized with the same strand of the genomic DNA at the two different binding sites. The linear distance between the two binding sites in the gene DNA established a repeating pattern of parallel polymeric nanocube chains spaced ˜480 nm apart. The ordered superstructure of nanocube chains provided an indication of the target gene. Non-limiting examples of detecting the ordered superstructure are diffraction of light passing through, or incident light reflected, from the sample and optical microscopy imaging. For instance, FIG. 10A shows a bright-field microscopy image of the assembled superstructure of parallel chains of the nanocubes described in this example at approximately 1000× magnification (see figures for scale bars). FIG. 10B contains the same oligonucleotide coated nanocubes as FIG. 10A, except the sample shown in FIG. 10B lacks the TUBB6 gene fragment required to assemble parallel arrays of nanocubes, in addition to lacking the oligonucleotide linkers necessary to form polymeric nanocube chains.

Hybridization of the single-stranded oligonucleotides on the nanocube surface to the target gene may require denaturation of the double-stranded genomic DNA. In this example, thermal denaturation for several minutes at 85° C. was used. To aid in the denaturation of the genomic DNA, several 15-nucleotide long, single-stranded oligonucleotides were hybridized to regions surrounding the binding sites complementary to the nucleotides sequences on the nanocubes, 5′-CGGAGACTGTCCATTGTCCCAGGCT (SEQ ID NO: 9) and 5′-GCTGCGTGAGCTCGGGGACTGTGAG (SEQ ID NO: 10). These oligonucleotides maintained the exposure of single-stranded genomic DNA in the genomic DNA sequences targeted by the nanocubes by hindering the reannealing of the genomic DNA.

### Example 2

The presence of genes can be detected via the assembly of nanorods into microscale structures that can be observed optically or by scattering or other techniques. Nascent genomic DNA is used to “glue” or attach the nanorods together (FIG. 4B). The basic method used in this example has the following steps: (1) selecting genes of interest as the target, (2) selecting at least two binding sites from that target sequence, (3) synthesizing large nanorods (e.g., near or above the diffraction limit), and coating them in sequences of single-stranded DNA that are complimentary to the binding sites defined in step 2, (4) combining the genetic sample in solution with the nanorods and thermal cycling once from 98° C. to the annealing temperature, and (5) detecting whether or not the nanorods have aggregated. In contrast to PCR, no costly, time-consuming enzymatic amplification is used during the process. Sample preparation to obtaining final results typically takes less than 10 minutes.

As an example, consider an assay for presence of TUBB6 in maize (Tubulin Beta 6). TUBB6 is a ubiquitous gene used as a positive control in many types of maize genetic assays. Two synthetic DNA oligonucleotide sequences were used in this assay:

(SEQ ID NO: 11)
TGG GAT GCC AAG AAC/iSp18//3ThioMC6-D/
(SEQ ID NO: 12)
/5ThioMC6-D//iSp18/GTG AGG AAG GAA GCT

These included three non-nucleotide modifications, which were incorporated into the oligonucleotides attached to the nanoparticle surface:
/iSp18/ . . . Spacer 18 is an 18-atom hexa-ethyleneglycol spacer to extend DNA sequence away from nanoparticle surface.
/5ThioMC6-D/ . . . single thiol attached to 5′ end of the oligonucleotide. The ether linkage contains six carbon aliphatic spacer between thiol and 5′ end.
/3ThioMC6-D/ . . . single thiol attached to 3′ end of the oligonucleotide. The ether linkage contains three carbon aliphatic spacer between thiol and 3′ end.

Two separate preparations of oligonucleotide-coated nanoparticles (one coated with only 3′-thiolated oligos and the other 5′-thiolated) were mixed together for the assay. The results from these assays can be seen in FIGS. 5A-5B.

The detection method is greatly simplified over PCR. Target sequences can be detected by simply counting the number of nanorod aggregates. Machine vision (MV) or other techniques can be used to automatically identify and count the number of aggregates that appear in an optical microscope image (FIG. 5). The MV algorithms are straightforward, e.g., using simple geometric features such as area, perimeter, or concavity to classify each object in a binarized image as either a rod or assembly. When a target gene is present in a sample, the algorithm detects a larger percentage of aggregates in the image. In contrast, when the gene is absent, detected assemblies are rare, forming e.g., when rods randomly happen to align on top of one another in the microscope image. The gene can thus be determined as present or absent based on the number of monomeric rods and/or fraction of aggregates in the image. Multiplexing many samples on a microfluidic cartridge could also allow the screening of multiple contaminating traits with only one sample preparation.

In principle, even a single copy of genomic DNA can be detected optically, provided it bonds nanorods together. However, there is a practical detection limit since nanorods in a single static image may—by random chance—overlap, giving the appearance of a binding event. One can reduce the probability of accidental overlap, for example, by reducing the nanorod concentration. A sample container comprising a sealed microfluidics device prevents evaporation of small sample volumes at the required elevated temperatures. The chamber can be made with multiple chambers to allow simultaneous detection of multiple genes. Smaller chambers can reduce power consumption during the single thermal ramp enough to allow battery operation, if desired. This device may thus be portable or handheld, e.g., the size of a large cellphone. The device may utilize simultaneous mixing, heating, and imaging. The ability to do all three simultaneously may allow for real-time temperature control, which may be useful for achieving the specificity required to detect single nucleotide polymorphisms (SNPs).

In this example, large-scale synthesis of polyvinylpyrrolidone (PVP)-coated silver nanorods was conducted. A 20 mL batch of highly concentrated nanorods, 10 microns long and 0.2 microns diameter, were synthesized in six hours by seeding a reduced solution of AgNO₃ with silver nanoparticles using conventional laboratory equipment. The oligonucleotides were attached to the nanoparticle surface through a thiol moiety by incubating the reduced oligonucleotides with the nanoparticles in a salt buffer overnight. The solution was incubated while vortexing to prevent nanoparticles from precipitating out of solution. A hexapolyethylene glycol (PEG) spacer located between the thiol and nucleotide sequence placed the sequence away from the nanoparticle to reduce interference from the nanoparticle surface.

This method incorporated PCR primer sequences into the nanoparticle-linked oligonucleotides (FIG. 4). The nucleotide sequence on the nanoparticle surface was designed to hybridize to a complementary sequence in genomic DNA. Two different batches of nanoparticles were used to detect a genomic sequence. One nanoparticle was coated in a sequence complementary to a region within the target sequence and the other sequence was complementary to a site upstream or downstream from the first sequence. Both nanoparticle-coated sequences hybridized to the same strand of genomic DNA, so the genomic DNA connected the two nanoparticles in close proximity. To facilitate hybridization of the nanoparticles to genomic DNA, the genomic DNA was denatured by heating to 98° C. for one minute before cooling to an annealing temperature 5° C. below the melting temperature (Tₘ) of the nanoparticle sequences. To aid denaturation of the genomic DNA, blocking oligonucleotides were incorporated in the assay. The blocking oligonucleotides were 20-30 nucleotide single-stranded sequences that hybridized to the genomic DNA at sites near the primary target sequence to prevent re-annealing. The blocking oligonucleotides were designed to have a melting temperature of 10° C. above the target sequences, and were present in 1000-fold stoichiometric excess over the genomic DNA.

The specificity of the nanoparticle-attached sequence was found to be dependent on temperature. At low temperatures, non-specific hybridization occurred in strands that provide only partial sequence complementarity. To image the nanoparticles at a temperature 5° C. below the Tₘ of the sequence, a custom instrument was constructed that precisely controlled sample temperature with a Peltier thermoelectric element placed in contact with the top of the sample container. Positioned below the sample, a motorized microscope with an extra-long working distance objective lens imaged the nanoparticles. Raw image data was output over a USB for processing on an external computer. To improve the mixing of the large nanoparticles, a solenoid was actuated against the sample container at 50 Hz.

High-sensitivity detection of even a small number of genomic copies was obtained. Each nanoparticle was categorized as either an assembly of nanoparticles or monomeric. Nanoparticle assemblies corresponded to the presence of at least one genomic DNA copy linking nanoparticles together. This technique does not need complex fractionation of sample into thousands of separate containers, unlike certain other techniques. The 10 micron-long nanorods were easily distinguishable by brightfield and darkfield microscopy under magnifications as low as 100×. Thousands of nanoparticles could be imaged simultaneously and classified as monomer or assembly. Typically, 100 nucleotides separated the nanoparticle binding sites on the genomic DNA strand. The short distance made the nanoparticles appear to be in direct contact. An image-processing algorithm was developed that classified each nanoparticle in the image as either monomeric or assembled. The ratio of gene presence to absence correlated to the number of target genomic sequences in the sample. To quantify the percentage contaminating gene (e.g. a gene edited or genetically modified sequence) in a sample (e.g. a batch of seeds), a ubiquitous housekeeping genetic sequence was quantified from the same sample. The percentage contamination is the number of engineered sequence copies divided by the total genomic copies measured with the housekeeping sequence.

Microscope images were taken on 400× zoom with a 1.2 megapixel camera (FIGS. 5A and B). Images were imported in into Wolfram Mathematica version 11.2. Images were first sharpened over a pixel radius of 5 before applying a morphological binarization that converted all image pixel values black or white (or equivalently, 0 or 1). All connected white pixels were labeled as individual components, after which the geometrical components of each object could be measured. Objects with no holes, less than 100 pixels, and a caliper length to width ratio greater than 2 were classified as monomeric rods. Objects with a caliper width greater than 2 pixels and a total number of pixels greater than 100 were labeled as assemblies. These thresholds were chosen to maximize the agreement of between monomeric rods and assemblies that were classified via the algorithm with those that were classified by eye. Thresholds may be altered for use on different cameras. Similar results can be obtained using different metrics and thresholds (e.g. an objects perimeter or best fit ellipse). Likewise, hand-labeling objects and using various machine learning algorithms (e.g. neural networks, random forests, logistic regression, etc.) provided similar results.

While several embodiments of the present invention have been described and illustrated herein, those of ordinary skill in the art will readily envision a variety of other means and/or structures for performing the functions and/or obtaining the results and/or one or more of the advantages described herein, and each of such variations and/or modifications is deemed to be within the scope of the present invention. More generally, those skilled in the art will readily appreciate that all parameters, dimensions, materials, and configurations described herein are meant to be exemplary and that the actual parameters, dimensions, materials, and/or configurations will depend upon the specific application or applications for which the teachings of the present invention is/are used. Those skilled in the art will recognize, or be able to ascertain using no more than routine experimentation, many equivalents to the specific embodiments of the invention described herein. It is, therefore, to be understood that the foregoing embodiments are presented by way of example only and that, within the scope of the appended claims and equivalents thereto, the invention may be practiced otherwise than as specifically described and claimed. The present invention is directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the scope of the present invention.

In cases where the present specification and a document incorporated by reference include conflicting and/or inconsistent disclosure, the present specification shall control. If two or more documents incorporated by reference include conflicting and/or inconsistent disclosure with respect to each other, then the document having the later effective date shall control.

All definitions, as defined and used herein, should be understood to control over dictionary definitions, definitions in documents incorporated by reference, and/or ordinary meanings of the defined terms.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of,” or “exactly one of.”

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

When the word “about” is used herein in reference to a number, it should be understood that still another embodiment of the invention includes that number not modified by the presence of the word “about.”

It should also be understood that, unless clearly indicated to the contrary, in any methods claimed herein that include more than one step or act, the order of the steps or acts of the method is not necessarily limited to the order in which the steps or acts of the method are recited.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03.

What is claimed is:

1. A composition, comprising:
a plurality of substantially identical first nanostructures each comprising a first plurality of nanoparticles joined by nucleic acids;
a plurality of substantially identical second nanostructures each comprising a second plurality of nanoparticles joined by nucleic acids; and
a plurality of target nucleic acids, at least some of which are immobilized to at least some of the plurality of first nanostructures and at least some of the plurality of second nanostructures to form a plurality of discrete, substantially identical molecular superstructures,
wherein each molecular superstructure of the plurality of discrete, substantially identical molecular superstructures comprises a target nucleic acid immobilized to both a first nanostructure and a second nanostructure.

2. The composition of claim 1, wherein at least some of the target nucleic acids has a length of at least 1,000 nt.

3. The composition of any one of claim 1 or 2 wherein at least some of the target nucleic acids comprise genomic DNA.

4. The composition of any one of claims 1-3, wherein the substantially identical molecular superstructures each comprise at least 5 target nucleic acids.

5. The composition of any one of claims 1-4, wherein the first nanostructure binds to a first site on a target nucleic acid and the second nanostructure binds to a second site on the target nucleic acid, wherein the first site and the second site are separated by at least 300 nm.

6. The composition of any one of claims 1-5, wherein the first nanostructure binds to a first site on a target nucleic acid and the second nanostructure binds to a second site on the target nucleic acid, wherein the first site and the second site are separated by at least 1,000 nt.

7. The composition of any one of claims 1-6, wherein the substantially identical molecular superstructures each comprise at least 5 nanostructures.

8. The composition of any one of claims 1-7, wherein the substantially identical molecular superstructure each comprise a repeating arrangement of nanostructures.

9. The composition of any one of claims 1-8, wherein the substantially identical molecular superstructures have a dynamic spacing between nanostructures of at least 300 nm.

10. The composition of any one of claims 1-9, wherein the substantially identical molecular superstructures have a dynamic spacing between the nanostructures that diffracts visible light.

11. The composition of any one of claims 1-10, wherein the first nanostructure has a largest internal dimension of at least 300 nm.

12. The composition of any one of claims 1-11, wherein at least some nanoparticles of the first plurality of nanoparticles are nanocubes that are substantially cubical.

13. The composition of any one of claims 1-12, wherein the first nanostructure comprises at least 5 nanoparticles.

14. The composition of any one of claims 1-13, wherein the nanoparticles of the first plurality of nanoparticles have an average largest internal dimension of at least about 100 nm.

15. The composition of any one of claims 1-14, wherein the nanoparticles of the first plurality of nanoparticles are joined in face-to-face contact.

16. The composition of claim 15, wherein the face-to-face contact between the nanoparticles is defined by binding interactions between the respective contacting nanoparticles.

17. The composition of any one of claims 1-16, wherein the first nanostructure is immobilized relative to a surface.

18. The composition of any one of claims 1-17, wherein the first nanostructure is immobilized relative to a surface via a tether.

19. A composition, comprising:
a plurality of substantially identical first nanostructures each comprising a first plurality of self-assembled nanoparticles;
a plurality of substantially identical second nanostructures each comprising a second plurality of self-assembled nanoparticles; and
a plurality of target molecules, at least some of which are immobilized to at least some of the plurality of first nanostructures and at least some of the plurality of second nanostructures to form a plurality of discrete, substantially identical molecular superstructures,
wherein each molecular superstructure of the plurality of discrete, substantially identical molecular superstructures comprises a target molecule immobilized to both a first nanostructure and a second nanostructure.

20. The composition of claim 19, wherein at least some of the target molecules comprise a polymer.

21. The composition of any one of claim 19 or 20, wherein at least some of the target molecules comprise a nucleic acid.

22. The composition of claim 21, wherein the nucleic acid comprises DNA.

23. The composition of any one of claim 21 or 22, wherein the nucleic acid has a length of at least 1,000 nt.

24. The composition of any one of claims 21-23, wherein the nucleic acid has a length of at least 1,000,000 nt.

25. The composition of any one of claims 21-24 wherein at least some of the target molecules comprise genomic DNA.

26. The composition of any one of claims 19-25, wherein at least some of the target molecules comprise a carbohydrate.

27. The composition of any one of claims 19-26, wherein at least some of the target molecules comprise a protein.

28. The composition of any one of claims 19-27, wherein at least some of the target molecules have a length of at least about 20 nm.

29. The composition of any one of claims 19-28, wherein the first nanostructure binds to a first site on one of the target molecules and the second nanostructure binds to a second site on the target molecule, wherein the first site and the second site are separated by at least 10 nm.

30. The composition of claim 29, wherein the first site and the second site are separated by at least 100 nm.

31. The composition of any one of claim 29 or 30, wherein the first site and the second site are separated by at least 300 nm.

32. The composition of any one of claims 19-31, wherein the substantially identical molecular superstructures each comprise at least 5 target molecules.

33. The composition of any one of claims 19-32, wherein the substantially identical molecular superstructures each comprise at least 10 target molecules.

34. The composition of any one of claims 19-33, wherein the substantially identical molecular superstructure each comprise at least 5 target molecules immobilized to both the first nanostructure and the second nanostructure.

35. The composition of any one of claims 19-34, wherein the substantially identical molecular superstructures each further comprise a third nanostructure comprising a third plurality of self-assembled nanoparticles.

36. The composition of claim 35, wherein the one or more target molecules is further immobilized to the third nanostructure to form the molecular superstructures.

37. The composition of any one of claim 35 or 36, further comprising one or more second target molecules immobilized to both the second nanostructure and the third nanostructure.

38. The composition of any one of claims 19-37, wherein the substantially identical molecular superstructures each comprises at least 5 nanostructures.

39. The composition of any one of claims 19-38, wherein the substantially identical molecular superstructures each comprises at least 10 nanostructures.

40. The composition of any one of claims 19-39, wherein the substantially identical molecular superstructures each comprises a repeating arrangement of nanostructures.

41. The composition of any one of claims 19-40, wherein the substantially identical molecular superstructures have a dynamic spacing between the repeating nanostructures of at least 10 nm.

42. The composition of claim 41, wherein the dynamic spacing between the repeating nanostructures is at least 100 nm.

43. The composition of any one of claim 41 or 42, wherein the dynamic spacing between the repeating nanostructures is at least 300 nm.

44. The composition of any one of claims 41-43, wherein the dynamic spacing between the repeating nanostructures is less than 1,000 nm.

45. The composition of any one of claims 41-44, wherein the substantially identical molecular superstructures have a dynamic spacing between the repeating nanostructures that diffracts light.

46. The composition of any one of claims 41-45, wherein the substantially identical molecular superstructure have a dynamic spacing between the repeating nanostructures that diffracts visible light.

47. The composition of any one of claims 41-46, wherein the substantially identical molecular superstructure have a dynamic spacing between the repeating nanostructures that diffracts ultraviolet light.

48. The composition of any one of claims 41-47, wherein the substantially identical molecular superstructures have a dynamic spacing between the repeating nanostructures that scatters light.

49. The composition of any one of claims 19-48, wherein the first nanostructure has a largest internal dimension of at least 300 nm.

50. The composition of any one of claims 19-49, wherein at least some nanoparticles of the first plurality of nanoparticles are polyhedral nanoparticles.

51. The composition of any one of claims 19-50, wherein at least some nanoparticles of the first plurality of nanoparticles are nanocubes that are substantially cubical.

52. The composition of any one of claims 19-51, wherein the first nanostructure comprises nanoparticles defining a structure comprising at least 2 nanocubes.

53. The composition of any one of claims 19-52, wherein the first nanostructure comprises nanoparticles defining a substantially planar structure.

54. The composition of any one of claims 19-53, wherein the first nanostructure comprises nanoparticles defining a substantially non-planar structure.

55. The composition of any one of claims 19-54, wherein at least some nanoparticles of the first plurality of nanoparticles are substantially cylindrical.

56. The composition of any one of claims 19-55, wherein at least some nanoparticles of the first plurality of nanoparticles are nanorods.

57. The composition of any one of claims 19-56, wherein the first nanostructure comprises at least 3 nanoparticles.

58. The composition of any one of claims 19-57, wherein the first nanostructure comprises at least 5 nanoparticles.

59. The composition of any one of claims 19-58, wherein the first nanostructure comprises at least 10 nanoparticles.

60. The composition of any one of claims 19-59, wherein the first nanostructure comprises nanoparticles arranged topologically linearly.

61. The composition of any one of claims 19-60, wherein the first nanostructure comprises linearly arranged nanoparticles.

62. The composition of any one of claims 19-61, wherein the nanoparticles of the first plurality of nanoparticles have an average largest internal dimension of less than about 10 micrometers.

63. The composition of any one of claims 19-62, wherein the nanoparticles of the first plurality of nanoparticles have an average largest internal dimension of at least about 100 nm.

64. The composition of any one of claims 19-63, wherein the nanoparticles of the first plurality of nanoparticles are joined in face-to-face contact.

65. The composition of claim 64, wherein the face-to-face contact between the nanoparticles is defined by binding interactions between the respective contacting nanoparticles.

66. The composition of claim 65, wherein each of the binding interactions within the first nanostructure comprises no more than 10% of the total binding interactions within the first nanostructure.

67. The composition of any one of claim 65 or 66, wherein at least some of the binding interactions are specific binding interactions.

68. The composition of any one of claims 65-67, wherein at least some of the binding interactions are nucleic acid interactions.

69. The composition of any one of claims 65-68, wherein at least some of the binding interactions are hydrogen bond interactions.

70. The composition of any one of claims 65-69, wherein at least some of the binding interactions are covalent couplings.

71. The composition of any one of claims 65-70, wherein at least some of the binding interactions are hydrophobic interactions.

72. The composition of any one of claims 19-71, wherein at least some nanoparticles of the first plurality of nanoparticles comprise a metal.

73. The composition of any one of claims 19-72, wherein at least some nanoparticles of the first plurality of nanoparticles comprise gold.

74. The composition of any one of claims 19-73, wherein at least some nanoparticles of the first plurality of nanoparticles comprise copper.

75. The composition of any one of claims 19-74, wherein at least some nanoparticles of the first plurality of nanoparticles comprise a semiconductor.

76. The composition of any one of claims 19-75, wherein at least some nanoparticles of the first plurality of nanoparticles comprise silicon.

77. The composition of any one of claims 19-76, wherein each of nanoparticles of the first plurality of nanoparticles comprises a unique arrangement of nucleic acids.

78. The composition of any one of claims 19-77, wherein the substantially identical molecular superstructures are contained within a suspension.

79. The composition of any one of claims 19-78, wherein the first nanostructure is immobilized relative to a surface.

80. The composition of claim 79, wherein the first nanostructure is immobilized relative to a surface via a tether.

81. The composition of claim 80, wherein the tether comprises polyethylene glycol.

82. The composition of claim 80 or 81, wherein the tether comprises a polymer.

83. The composition of claim 80-82, wherein the tether comprises a nylon

84. The composition of claim 80-83, wherein the tether comprises a polypeptide.

85. The composition of claim 80-84, wherein the second nanostructure is immobilized relative to the surface.

86. A composition, comprising:
a first nanostructure comprising a first plurality of nanoparticles joined by nucleic acids;
a second nanostructure comprising a second plurality of self-assembled joined by nucleic acids; and
a plurality of target nucleic acids each immobilized to both the first nanostructure and the second nanostructure to form a molecular superstructure, wherein the molecular superstructure has a dynamic spacing between the first and second nanostructures of at least 200 nm.

87. A method of determining a target molecule, comprising:
exposing a sample suspected of comprising a target molecule to a suspension comprising a first nanostructure comprising a first plurality of nanoparticles joined by nucleic acids, and a second nanostructure comprising a second plurality of nanoparticles joined by nucleic acids; and
determining binding of the target molecule to both the first nanostructure and the second nanostructure, wherein binding of the target molecule to both the first nanostructure and the second nanostructure forms a molecular superstructure comprising the first nanostructure, the second nanostructure, and the target molecule.

88. The method of claim 87, wherein determining binding of the target molecule comprises determining diffraction of light within the sample.

89. The method of any one of claim 87 or 88, wherein determining binding of the target molecule comprises determining diffraction of visible light within the sample.

90. The method of any one of claims 87-89, wherein determining binding of the target molecule comprises determining diffraction of ultraviolet light within the sample.

91. The method of any one of claims 87-90, wherein determining binding of the target molecule comprises determining scattering of light within the sample.

92. The method of any one of claims 87-91, wherein determining binding of the target molecule comprises applying coherent light to at least a portion of the sample.

93. The method of any one of claims 87-92, wherein determining binding of the target molecule comprises applying laser light to at least a portion of the sample.

94. The method of any one of claims 87-93, wherein determining binding of the target molecule comprises optically determining the molecular superstructure within the sample.

95. The method of claim 94, comprising determining the molecular superstructure within the sample with the unaided eye.

96. The method of any one of claim 94 or 95, comprising determining the molecular superstructure within the sample using a microscope.

97. The method of claim 96, comprising determining the molecular superstructure within the sample using an optical microscope.

98. The method of any one of claim 96 or 97, comprising determining the molecular superstructure within the sample using an electron microscope.

99. The method of any one of claims 96-98, comprising determining the molecular superstructure within the sample using an atomic force microscope.

100. A method of determining a target molecule, comprising:
exposing a sample suspected of comprising a target molecule to a suspension comprising a first nanostructure comprising self-assembled nanoparticles, and a second nanostructure comprising self-assembled nanoparticles; and
determining binding of the target molecule to both the first nanostructure and the second nanostructure.

101. A device, comprising:
a substrate comprising a plurality of chambers, at least some chambers comprising a first nanostructure comprising a first plurality of nanoparticles joined by nucleic acids, a second nanostructure comprising a second plurality of self-assembled joined by nucleic acids; and
a source of coherent light positioned to direct coherent light at at least one chamber of the plurality of chambers.

102. The device of claim 101, further comprising a detector positioned to detect light from the at least one chamber in which the coherent light from the source of coherent light is directed.

103. A device, comprising:
a substrate comprising a plurality of chambers, at least some chambers comprising a first nanostructure comprising a first plurality of nanoparticles joined by nucleic acids, a second nanostructure comprising a second plurality of self-assembled joined by nucleic acids;
a source of light positioned to direct light at at least one chamber of the plurality of chambers; and
a detector positioned to detect scattered and/or diffracted light from the at least one chamber in which the light from the source of light is directed.

104. A method of determining a target molecule, comprising:
exposing a sample suspected of containing a target molecule to two or more nanoparticles that self-assemble with the target molecule to form a molecular superstructure comprising the two or more nanoparticles and the target molecule; and
determining the molecular superstructure within the sample.

105. The method of claim 104, wherein at least some of the target molecules comprise genomic DNA.

106. The method of any one of claim 104 or 105, wherein at least some of the nanoparticles are rods.

107. The method of any one of claims 104-106, wherein at least some of the nanoparticles are cubes.

108. The method of any one of claims 104-107, wherein at least some of the nanoparticles are spheres.

109. The method of any one of claims 104-108, wherein at least some of the molecular superstructures are X-shaped, L-shaped, S-shaped, Z-shaped, and/or zigzags.

110. The method of any one of claims 104-109, wherein determining the molecular superstructure within the sample comprises determining the molecular superstructure within the sample using a microscope.

111. The method of any one of claims 104-110, wherein determining the molecular superstructure within the sample comprises determining the molecular superstructure within the sample using an optical microscope.

112. The method of any one of claims 104-111, wherein determining the molecular superstructure within the sample comprises acquiring an image, and determining the molecular superstructure using image processing of the image.

113. The method of any one of claims 104-112 wherein determining the molecular superstructure within the sample comprises determining fluorescence of the molecular superstructure.

114. A method of quantifying the amount of a target molecule, comprising:
exposing a sample suspected of containing a target molecule to two or more nanoparticles that self-assemble with the target molecule to form a molecular superstructure comprising the two or more nanoparticles and the target molecule;
determining the molecular superstructures within the sample; and
quantifying the target molecules using the distribution of nanoparticles and molecular superstructures observed.

115. The method of claim 114, wherein at least some of the target molecules comprise genomic DNA.

116. The method of any one of claim 114 or 115, wherein at least some of the nanoparticles are rods.

117. The method of any one of claims 114-116, wherein at least some of the nanoparticles are cubes.

118. The method of any one of claims 114-117, wherein at least some of the nanoparticles are spheres.

119. The method of any one of claims 114-118, wherein at least some of the molecular superstructures are X-shaped, L-shaped, S-shaped, Z-shaped, and/or zigzags.

120. The method of any one of claims 114-119, wherein determining the molecular superstructure within the sample comprises determining the molecular superstructure within the sample using a microscope.

121. The method of any one of claims 114-120, wherein determining the molecular superstructure within the sample comprises determining the molecular superstructure within the sample using an optical microscope.

122. The method of any one of claims 114-121, wherein determining the molecular superstructure within the sample comprises acquiring an image, and determining the molecular superstructure using image processing of the image.

123. The method of any one of claims 114-122 wherein determining the molecular superstructure within the sample comprises determining fluorescence of the molecular superstructure.
